(19) United States
(12) Patent Application Publication
(10) Pub. No.: US 2005/0129772 A1
(43) Pub. Date: Jun. 16, 2005

(54) Compositions comprising an HIV protease inhibitor

(75) Inventors: Scott Wendell Smith, San Diego, CA (US); Dwayne Thomas Friesen, Bend, OR (US); Douglas Alan Lorenz, Bend, OR (US); David Keith Lyon, Bend, OR (US); Rodney James Ketner, Bend, OR (US); James Blair West, Bend, OR (US)

Correspondence Address: AGOURON PHARMACEUTICALS, INC., 10350 NORTH TORREY PINES ROAD, LA JOLLA, CA, 92037, US

(73) Assignee: Agouron Pharmaceuticals, Inc.

(21) Appl. No.: 11/008,325

(22) Filed: Dec. 8, 2004

Related U.S. Application Data
(60) Provisional application No. 60/528,381, filed on Dec. 9, 2003.
(60) Provisional application No. 60/542,352, filed on Feb. 6, 2004.
(60) Provisional application No. 60/610,123, filed on Sep. 14, 2004.

(51) Int. Cl. A61K 31/426; C07D 277/18; A61K 9/14

(52) U.S. Cl. 424/488; 514/370; 548/190

(57) ABSTRACT

The present invention relates to pharmaceutical compositions comprising amorphous (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, their methods of preparation, their use in inhibiting the HIV protease enzyme, and their use in treating HIV-infected mammals, such as humans.

This application claims priority under 35 U.S.C. § 119(e) to U.S. Provisional Application No. 60/528,381, filed Dec. 9, 2003, 60/542,352, filed Feb. 6, 2004, and 60/610,123, filed Sep. 14, 2004.

### BACKGROUND

The compound (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide (also called “(R)-3-((2S,3S)-2-hydroxy-3-{[1-(3-hydroxy-2-methyl-phenyl)-methanoyl]-amino}-4-phenyl-butanoyl)-5,5-dimethyl-thiazolidine-4-carboxylic acid allylamide,” “(4R)-3-[(2S,3S)-2-hydroxy-3-(3-hydroxy-2-methyl-benzoylamino)-4-phenyl-butyryl]-5,5-dimethyl-thiazolidine-4-carboxylic acid allylamide,” or “4-thiazolidinecarboxamide, 3-[(2S,3S)-2-hydroxy-3-[(3-hydroxy-2-methylbenzoyl)amino]-1-oxo-4-phenylbutyl]-5,5-dimethyl-N-2-propenyl-, (4R)-,” and hereinafter referred to as “Compound A”) is an inhibitor of the HIV protease enzyme that may be used for the treatment of HIV-infected mammals, such as humans. Compound A and its preparation are disclosed in U.S. patent application Ser. Nos. 10/166,979 filed Jun. 11, 2002; 60/527,477, filed Dec. 4, 2003, and, 10/782,602, filed Dec. 4, 2003, all of which incorporated herein by reference for this purpose. Crystalline Compound A is very slightly soluble in aqueous solution, having an aqueous solubility of about 0.16 mgA/mL, 0.14 mgA/mL, and 0.16 mgA/mL in unbuffered water (pH 8.3), normal saline (pH 8.2) and 0.1 N HCl (pH 1.2), respectively (temperature of 26° C.). This low aqueous solubility, combined with a low in vivo permeability, result in low oral bioavailability for crystalline Compound A.

Accordingly, there is a need to improve the bioavailability of Compound A, while maintaining the stability of Compound A in a dosage form.

### SUMMARY

The invention provides a pharmaceutical composition comprising amorphous Compound A. Amorphous Compound A has improved solubility relative to crystalline Form I of Compound A, and when orally administered to a mammal, such as a human, provides improved bioavailability relative to crystalline Form I.

One aspect of the present invention provides amorphous Compound A, or a pharmaceutically acceptable salt or solvate thereof.

Another aspect of the present invention provides pharmaceutical compositions comprising amorphous Compound A, or a pharmaceutically acceptable salt or solvate thereof.

In still another aspect of the present invention are provided pharmaceutical compositions comprising Compound A, or a pharmaceutically acceptable salt or solvate thereof, wherein at least about 5 wt % of the total amount of Compound A present is in an amorphous form. Alternatively, pharmaceutical compositions are provided comprising Compound A, or a pharmaceutically acceptable salt or solvate thereof, wherein at least about 10 wt %, or at least about 15 wt % or at least about 20 wt %, or at least about 30 wt %, or at least about 40 wt %, or at least about 50 wt/o, or at least about 60 wt %, or at least about 70 wt/o, or at least about 80 wt %, or at least about 90 wt %, or at least about 95 wt % of the total amount of Compound A present is in an amorphous form.

In one aspect, the pharmaceutical compositions comprise (1) amorphous Compound A, or a pharmaceutically acceptable salt or solvate thereof, and (2) a matrix.

In one embodiment, the pharmaceutical composition comprises (1) amorphous Compound A and (2) a matrix comprising a concentration-enhancing polymer. The concentration-enhancing polymer further improves the concentration of dissolved Compound A in a use environment. It has been found that only relatively small amounts of polymer are needed to sustain the high dissolved drug concentration provided by amorphous Compound A. When amorphous Compound A is administered alone to a use environment, it provides an initially enhanced dissolved concentration of Compound A that decreases over time to the lower equilibrium concentration provided by crystalline Form I. However, compositions comprising a concentration-enhancing polymer provide not only an initially enhanced dissolved concentration, but also a sustained dissolved concentration over a physiologically relevant time. In fact, only small amounts of polymer, ranging from about 25 wt/o to even about 1 wt % of the composition, result in substantial improvements in dissolution performance of Compound A relative to not only crystalline Form I Compound A alone, but also amorphous Compound A alone.

In still another aspect are provided pharmaceutical compositions, comprising Compound A, or a pharmaceutically acceptable salt or solvate thereof, and a matrix, wherein said matrix comprises at least one of an ionizable cellulosic polymer, a nonionizable cellulosic polymer, or a noncellulosic polymer.

In still further aspects, the at least one ionizable cellulosic polymer is selected from at least one of hydroxypropyl methyl cellulose acetate succinate, carboxymethyl ethyl cellulose, cellulose acetate phthalate, hydroxypropyl methyl cellulose phthalate, methyl cellulose acetate phthalate, cellulose acetate trimellitate, hydroxypropyl cellulose acetate phthalate, hydroxypropyl methyl cellulose acetate phthalate, cellulose acetate terephthalate and cellulose acetate isophthalate, and mixtures thereof.

Still further are provided such compositions wherein the at least one nonionizable, cellulosic polymer is selected from hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose, and mixtures thereof.

Other aspects provide such compositions wherein said at least one non-cellulosic polymer is selected from carboxylic acid functionalized polymethacrylates, carboxylic acid functionalized polyacrylates, amine-functionalized polyacrylates, amine-fuctionalized polymethacrylates, proteins, carboxylic acid functionalized starches, vinyl polymers and copolymers having at least one substituent selected from the group consisting of hydroxyl, alkylacyloxy, and cyclicamido, vinyl copolymers of at least one hydrophilic, hydroxyl-containing repeat unit and at least one hydrophobic, alkyl- or aryl-containing repeat unit, polyvinyl alcohols that have at least a portion of their repeat units in the unhydrolyzed form, polyvinyl alcohol polyvinyl acetate copolymers, polyethylene glycol polypropylene glycol copolymers, polyvinyl pyrrolidone, polyethylene polyvinyl alcohol copolymers, polyoxyethylene-polyoxypropylene block copolymers and mixtures thereof.

In another aspect of the present invention are provided pharmaceutical compositions comprising Compound A, or a pharmaceutically acceptable salt or solvate thereof, and a matrix, wherein at least about 5 wt % of the total amount of Compound A present is in an amorphous form. Alternatively, pharmaceutical compositions are provided comprising Compound A, or a pharmaceutically acceptable salt or solvate thereof, and a matrix, wherein at least about 10 wt %, or at least about 15 wt/o or at least about 20 wt %, or at least about 30 wt %, or at least about 40 wt %, or at least about 50 wt %, or at least about 60 wt %, or at least about 70 wt %, or at least about 80 wt %, or at least about 90 wt %, or at least about 95 wt % of the total amount of Compound A present is in an amorphous form.

Another aspect provides pharmaceutical compositions comprising (1) Compound A and (2) a matrix, wherein at least a portion of Compound A is amorphous.

In another embodiment, the pharmaceutical composition comprises a solid amorphous dispersion comprising Compound A, or a pharmaceutically acceptable salt or solvate thereof, and a matrix. In one aspect, the solid amorphous dispersion comprises at least about 30 wt % Compound A, or pharmaceutically acceptable salt or solvate thereof. In another aspect, the solid amorphous dispersion comprises at least about 40 wt %, at least about 50 wt %, at least about 60 wt %, at least about 70 wt %, at least about 80 wt %, at least about 90 wt %, or at least about 95 wt % of Compound A, or a pharmaceutically acceptable salt or solvate thereof.

In another embodiment, the invention provides a pharmaceutical composition comprising Compound A, that when administered to an in vitro aqueous environment, provides at least one of (a) a maximum dissolved concentration of Compound A in the use environment that is at least about 1.25-fold that provided by a control composition; and (b) a concentration of Compound A in the use environment versus time area under the curve (AUC) for any period of at least 90 minutes between the time of introduction into the use environment and about 270 minutes following introduction to the use environment that is at least about 1.25-fold that of the control composition. The control composition consists essentially of an equivalent quantity of Compound A in crystalline Form I alone. In another embodiment, the use environment discussed above consists essentially of 20 mM Na₂HPO₄, 47 mM KH₂PO₄, 87 mM NaCl, and 0.2 mM KCl, at pH 6.5, and 290 mOsm/kg, and at a temperature 37° C., wherein the total amount of said use environment is about 1.8 mL and the amount of Compound A used is such that the total concentration of Compound A would have been 3000 μg/mL if all of Compound A had dissolved. In another aspect, such a pharmaceutical composition comprises Compound A, wherein at least a portion of Compound A is in an amorphous form. In still other aspects are provided such compositions wherein at least about 5 wt % of the total amount of Compound A present is in an amorphous form. In still other aspects are provided such compositions wherein at least about 10 wt %, or at least about 15 wt % or at least about 20 wt %, or at least about 30 wt %, or at least about 40 wt %, or at least about 50 wt %, or at least about 60 wt %, or at least about 70 wt %, or at least about 80 wt %, or at least about 90 wt %, or at least about 95 wt % of the total amount of Compound A present is in an amorphous form.

In another aspect of the present invention are provided methods of achieving a plasma concentration of Compound A in a mammal, such as a human, of from about 0.001 μM to about 5 μM, said method comprising administering to said mammal a sufficient amount of a pharmaceutical composition comprising Compound A alone, or a pharmaceutically acceptable salt or solvate thereof, or in combination with a matrix. In one embodiment, the composition comprises amorphous Compound A. In another embodiment, the composition comprises amorphous Compound A and at least one matrix. In another aspect of the present invention, the plasma concentration of Compound A in a mammal, such a human, is in the range of from about 0.01 μM to about 2.5 μM, or from about 0.02 μM to about 1 μM, or from about 0.025 μM to about 1 μM, or from about 0.05 μM to about 1 μM.

In another aspect of the present invention are provided such methods wherein said plasma concentrations of Compound A in a mammal, such as a human, are maintained for at least about 6 hours after said administration, or at least about any of 8, 10, 12, 14, 16, 18, 20, 22, or 24 hours after said administration.

In a still further aspect of the present invention are provided methods of achieving an average plasma concentration of Compound A in the plasma of a mammal, such as a human, in the range of from about 0.001 μM to about 2.5 μM for about 6 to about 24 hours, the method comprising administering to said mammal a sufficient amount of a pharmaceutical composition comprising Compound A alone, or a pharmaceutically acceptable salt or solvate thereof, or in combination with a matrix. In one embodiment, the composition comprises amorphous Compound A. In another embodiment, the composition comprises amorphous Compound A and at least one matrix. In other aspects of the present invention, the average plasma concentration in a mammal of Compound A is in the range of from about 0.02 μM to about 1 μM, or from about 0.025 μM to about 1 μM, or from about 0.05 μM to about 1 μM. In another aspect of the present invention such average plasma concentrations for such times are achieved by administering to said mammal a dose of a pharmaceutical composition comprising Compound A alone, or a pharmaceutically acceptable salt or solvate thereof, or in combination with a matrix, wherein said dose of Compound A is in the range of from about 300 mgA to about 3600 mgA of Compound A in said composition. In one embodiment, such compositions comprise amorphous Compound A. In another embodiment, such compositions comprise amorphous Compound A and at least one matrix. By “mgA” is meant the milligrams of active Compound A. The present invention also provides such methods wherein the dose of Compound A is about 400 mgA, 600 mgA, 800 mgA, 1000 mgA, 1200 mgA, 1400 mgA, 1600 mgA, 1800 mgA, 2000 mgA, 2200 mgA, 2400 mgA, 2600 mgA, 2800 mgA, 3000 mgA, 3200 mgA, or 3400 mgA.

In yet another aspect of the present invention are provided methods of treating an HIV-infected mammal, such as a human, comprising administering to said HIV-infected mammal an HIV replication-inhibiting amount of a pharmaceutical composition comprising amorphous Compound A alone, or a pharmaceutically acceptable salt or solvate thereof, or in combination with a matrix.

A still further aspect of the present invention provides methods of treating AIDS or AIDS-related complex in an HIV-infected mammal, such as a human, comprising administering to said mammal a pharmaceutical composition comprising an HIV replication-inhibiting amount of amorphous Compound A, or a pharmaceutically acceptable salt or solvate thereof, alone or in combination with a matrix.

The present invention also provides methods of inhibiting HIV protease activity in an HIV-infected mammal, such as a human, comprising administering to said mammal a pharmaceutical composition comprising an HIV replication-inhibiting amount of amorphous Compound A, or a pharmaceutically acceptable salt or solvate thereof, alone or in combination with a matrix.

In still a further aspect of the present invention are provided methods of treating HIV in an infected mammal, such as a human, comprising administering to said mammal a pharmaceutical composition comprising an HIV replication-inhibiting amount of amorphous Compound A, or a pharmaceutically acceptable salt or solvate thereof, alone or in combination with a matrix, and at least one additional therapeutic agent chosen from nucleoside HIV reverse transcriptase inhibitors, non-nucleoside HIV reverse transcriptase inhibitors, HIV protease inhibitors, HIV integrase inhibitors, HIV fusion inhibitors, immune modulators, CCR5 antagonists, and antiinfectives. In one aspect of the present invention, the amorphous Compound A and the at least one additional therapeutic agent are administered as part of the same pharmaceutical composition. In yet another aspect, the amorphous Compound A and the at least one additional therapeutic agent are administered simultaneously or sequentially.

The present invention also provides pharmaceutical compositions comprising a therapeutically effective amount of amorphous Compound A alone, or a pharmaceutically acceptable salt or solvate thereof, or in combination with a matrix, and at least one additional therapeutic agent chosen from nucleoside HIV reverse transcriptase inhibitors, non-nucleoside HIV reverse transcriptase inhibitors, HIV protease inhibitors, HIV integrase inhibitors, HIV fusion inhibitors, immune modulators, CCR5 antagonists, and antiinfectives.

The present invention also affords such methods wherein the matrix is selected from at least one of an ionizable cellulosic polymer, a nonionizable cellulosic polymer, and a noncellulosic polymer as described above.

In yet another aspect of the present invention are provided the above-described methods wherein the administration of the pharmaceutical composition takes place once, twice, or three times a day.

The present invention also provides methods of using amorphous Compound A, or a pharmaceutically acceptable salt or solvate thereof, alone or in combination with a matrix in the manufacture of a medicament for the treatment of HIV infection in an infected mammal, such as a human. In addition, the present invention provides methods of using amorphous Compound A, or a pharmaceutically acceptable salt or solvate thereof, alone or in combination with a matrix in the manufacture of a medicament for the treatment of AIDS or AIDS-related complex in an HIV-infected mammal, such as a human.

In still another aspect of the present invention are provided methods of achieving an average plasma concentration of Compound A in the plasma of a mammal, such as a human, in the range of from about 0.001 μM to about 2.5 μM for about 6 to about 24 hours, the method comprising administering to said mammal an inhibitor of the cytochrome P450 enzyme and sufficient amount of a pharmaceutical composition comprising amorphous Compound A alone, or a pharmaceutically acceptable salt or solvate thereof, or in combination with a matrix. The present invention also provides such methods wherein the cytochrome P450 enzyme is the 3A4 isoform. Also provided are such methods wherein the inhibitor of the cytochrome P450 enzyme is ritonavir or delavirdine.

In another aspect, the invention provides a method for making amorphous Compound A using solvent-based processes.

Also provided herein are methods for preparing pharmaceutical compositions comprising:
(a) dissolving a compound in a spray solution comprising at least one solvent; and
(b) rapidly evaporating said at least one solvent from said spray solution to afford an amorphous form of said compound;
   wherein said compound is (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof.

In another aspect are provided such methods wherein the spray solution further comprises a matrix.

Further provided herein are such methods wherein the matrix comprises at least one polymer selected from an ionizable cellulosic polymer, a nonionizable cellulosic polymer, and a noncellulosic polymer.

Also provided herein are such methods of preparing pharmaceutical compositions wherein said at least one solvent is selected from methanol and mixtures of water and methanol.

In another embodiment, the composition further comprises a stabilizing agent to improve the chemical stability of Compound A. The stabilizing agent may be a base or an anti-oxidant. In one preferred embodiment, the composition comprises a solid amorphous dispersion, the solid amorphous dispersion further comprising a stabilizing agent. By “improve the chemical stability” of Compound A is meant slowing the rate of degradation of Compound A into another chemical compound or compounds.

In yet another aspect of the invention, a composition comprising amorphous Compound A is packaged so as to reduce degradation of Compound A. The packaging may either limit exposure of Compound A to humidity or oxygen, or both.

The term “crystalline,” as used herein, means a particular solid form of a compound of the invention that exhibits long-range order in three dimensions. Material that is crystalline may be characterized by techniques known in the art such as powder x-ray diffraction (PXRD) crystallography, solid state NMR, or thermal techniques such as differential scanning calorimetry (DSC).

The term “amorphous,” as used herein, means a particular solid form of a compound of the invention that has essentially no order in three dimensions. The term “amorphous” is intended to include not only material which has essentially no order, but also material which may have some small degree of order, but the order is in less than three dimensions and/or is only over short distances. Amorphous material may be characterized by techniques known in the art such as powder x-ray diffraction (PXRD) crystallography, solid state NMR, or thermal techniques such as differential scanning calorimetry (DSC).

The term “control composition,” as used herein, refers to a composition consisting essentially of crystalline Form I of Compound A. It is to be understood that the control compositions used herein are free from other compounds or ingredients that would effect the solubility of crystalline Form I of Compound A in the use environments described herein.

The term “crystalline Form I of Compound A,” as used herein, means the crystalline form of (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide that is characterized by being free from an amorphous form of Compound A as far as can be determined by one of ordinary skill in the art using analytical methods such as powder x-ray diffraction (PXRD), differential scanning calorimetry (DSC), solid state NMR (ssNMR), and Raman IR spectroscopy (Raman). Furthermore, the crystalline Form I of Compound A is characterized by having a powder x-ray diffraction pattern that is similar to that provided as Control 1 in FIG. 1. By “similar” is meant that one of ordinary skill in the art comparing the pattern of Control 1 in FIG. 1 with another experimentally determined pattern of a composition comprising a crystalline form of Compound A would say that they are the same polymorphic form, taking into account the known variability in the intensities and the position of the lines in a typical powder x-ray diffraction pattern that can depend on the specific experimental conditions used to obtain the pattern.

As used here in, the term “at least a portion of Compound A is in an amorphous form” means that at least 5 wt %, or at least 10 wt % of the total amount of Compound A in the composition is in an amorphous form.

The term “equivalent quantity,” as used herein refers to molar quantities of Compound A, measured as the theoretical number of moles of parent compound, (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, present in a given composition. For example, for a given amount of a composition comprising a salt or solvate of Compound A, an equivalent quantity of crystalline Form I of Compound A would be calculated by determining the theoretical number of moles of Compound A present in the composition and using an amount of crystalline Form I of compound A that would afford the same theoretical number of moles of parent Compound A.

The terms “administration,” “administering,” “dosage,” and “dosing,” as used herein refer to the delivery of a compound, or a pharmaceutically acceptable salt or solvate thereof, or of a pharmaceutical composition containing the compound, or a pharmaceutically acceptable salt or solvate thereof, to a mammal such that the compound is absorbed into the serum or plasma of the mammal.

The terms “co-administration” or “co-administering,” as used herein, refer to the administration of a combination of a first compound and a compound of the present invention, or a pharmaceutically acceptable salt or solvate thereof, either alone or in as part of a pharmaceutically acceptable composition. Such co-administration can be performed such that the first compound and the compound of the present invention are part of the same composition or part of the same unitary dosage form. Co-administration also includes administering a first compound and a compound of the present invention separately, but as part of the same therapeutic regimen. The two components, if administered separately, need not necessarily be administered at essentially the same time, although they can be if so desired. Thus co-administration includes, for example, administering a first compound and a compound of the present invention as separate dosages or dosage forms, but at the same time. Co-administration also includes separate administration at different times and in any order.

A “solvate” is intended to mean a pharmaceutically acceptable solvate form of a specified compound that retains the biological effectiveness of such compound. Examples of solvates include, but are not limited to, compounds of the invention in combination with water, isopropanol, ethanol, methanol, dimethylsulfoxide (DMSO), ethyl acetate, acetic acid, ethanolamine, or mixtures thereof. A “pharmaceutically acceptable salt” is intended to mean a salt that retains the biological effectiveness of the free acids and bases of the specified derivative, containing pharmacologically acceptable anions, and is not biologically or otherwise undesirable. Examples of pharmaceutically acceptable salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edislyate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, γ-hydroxybutyrate, iodide, isobutyrate, isothionate, lactate, lactobionate, laurate, malate, maleate, malonate, mandelate, mesylate, metaphosphate, methane-sulfonate, methylsulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phospate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodode, and valerate salts.

As used herein, a “use environment” can be either the in vivo environment of the GI tract of a mammal, such as a mammal and particularly a human, or the in vitro environment of a test solution, such as phosphate buffered saline (PBS) or Model Fasted Duodenal (MFD) solution. Concentration enhancement may be determined through either in vivo tests or through in vitro dissolution tests. A composition of the present invention meets the concentration enhancement criteria in at least one of the above test environments.

The foregoing and other objectives, features, and advantages of the invention will be more readily understood upon consideration of the following detailed description of the invention.

### BRIEF DESCRIPTION OF THE DRAWINGS

FIG. 1 plots the powder x-ray diffractions of Example 14 of crystalline Form I of Compound A and a solid amorphous dispersion of the present invention, and shows that Compound A in the solid amorphous dispersion is not crystalline.

FIG. 2 plots the powder x-ray diffractions of Example 15 of crystalline Form I of Compound A and a solid amorphous dispersion of the present invention after storage at different conditions, and shows the solid amorphous dispersions are physically stable.

### DETAILED DESCRIPTION

Compound A is (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide (also called “(R)-3-((2S,3S)-2-Hydroxy-3-{[1-(3-hydroxy-2-methyl-phenyl)-methanoyl]-amino}-4-phenyl-butanoyl)-5,5-dimethyl-thiazolidine-4-carboxylic acid allylamide,” “(4R)-3-[(2S,3S)-2-hydroxy-3-(3-hydroxy-2-methyl-benzoylamino)-4-phenyl-butyryl]-5,5-dimethyl-thiazolidine-4-carboxylic acid allylamide,” or “4-thiazolidinecarboxamide, 3-[(2S,3S)-2-hydroxy-3-[(3-hydroxy-2-methylbenzoyl)amino]-1-oxo-4-phenylbutyl]-5,5-dimethyl-N-2-propenyl-, (4R)-,”). Compound A has the following structure: 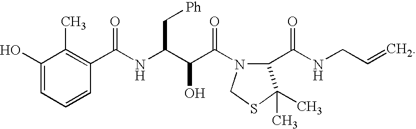
It has a molecular weight of 512, and crystalline Form I of Compound A has a melting point of about 176-178° C.

As used herein, the term “compound” is conventional, denoting the chemical species shown above and should be understood to include any pharmaceutically acceptable forms. By “pharmaceutically acceptable forms” is meant any pharmaceutically acceptable derivative or variation, including stereoisomers, stereoisomer mixtures, enantiomers, solvates, hydrates, isomorphs, polymorphs, pseudomorphs, neutral forms, salt forms and prodrugs.

### Amorphous Compound A

In one aspect, a composition comprises amorphous Compound A. By “amorphous” is meant that Compound A is not “crystalline.” By “crystalline” is meant that Compound A exhibits long-range order in three dimensions. Thus, the term amorphous is intended to include not only material which has essentially no order, but also material which may have some small degree of order, but the order is in less than three dimensions and/or is only over short distances. Amorphous material may be characterized by those of ordinary skill in the art using techniques known in the art such as powder x-ray diffraction (PXRD) crystallography, solid state NMR, Raman IR spectroscopy, or thermal techniques such as differential scanning calorimetry (DSC). While the compositions of the present invention may contain both amorphous and crystalline Compound A, it is also specifically contemplated that of the total amount of Compound A present in the compositions of the invention at least about 5 wt %, or at least about 10 wt %, or at least about 30 wt %, or at least about 40 wt %, or at least about 50 wt %, or at least about 60 wt %, or at least about 70 wt %, or at least about 80 wt %, or at least about 90 wt %, or at least about 95 wt %, may be in an amorphous form.

It has been found that an amorphous form Compound A provides improved concentration of dissolved Compound A in a use environment relative to crystalline Form I of Compound A. Typically, the amorphous Compound A achieves a higher maximum drug concentration (MDC) of Compound A in the use environment relative to a control composition consisting of an equivalent amount of crystalline Form I of Compound A. It is to be understood that the crystalline Form I of Compound A used as a control composition herein is free from solubilizers or other components that would materially affect the solubility of Compound A in an appropriate use environment. Preferably, an amorphous form of Compound A increases the maximum dissolved concentration (MDC) of Compound A in an aqueous use environment by at least about 1.25-fold, or at least 2-fold, or at least 3-fold, relative to the control composition, said control composition consisting essentially of crystalline Form I of Compound A. In addition, an amorphous form of Compound A may also increase the dissolution area under the concentration versus time curve (AUC) of Compound A in the environment of use relative to an equivalent amount of crystalline Form I of Compound A. The calculation of an AUC is a well-known procedure in the pharmaceutical arts and is described, for example, in Welling, “Pharmacokinetics Processes and Mathematics,” ACS Monograph 185 (1986). For example, an amorphous form of Compound A increases the AUC of Compound A in an aqueous use environment by at least about 1.25-fold, or at least 2-fold, or at least 3-fold, relative to the control composition consisting essentially of crystalline Form I of Compound A. For example, when tested in an in vitro use environment consisting of a phosphate buffered saline solution at 37° C., pH 6.5 and 290 mOsm/kg, an amorphous form of Compound A provided a maximum dissolved drug concentration (MDC) that was 10.3-fold that provided by crystalline Form I of Compound A, and an area under the dissolved drug concentration versus time curve for the initial ninety minutes after administration to the use environment (AUC₉₀) that was 8.3-fold that provided by crystalline Form I of Compound A.

Where the use environment is the GI tract of an animal, dissolved drug concentration may be determined by any conventional method known in the art. One method is a deconvolution method. In this method, the serum or plasma drug concentration is plotted along the ordinate (y-axis) against the blood sample time along the abscissa (x-axis). The data may then be analyzed to determine drug release rates in the GI tract using any conventional analysis, such as the Wagner-Nelson or Loo-Riegelman analysis. See also Welling, “Pharmacokinetics: Processes and Mathematics” (ACS Monograph 185, Amer. Chem. Soc., Washington, D.C., 1986). Treatment of the data in this manner yields an apparent in vivo drug release profile. Another method is to intubate the patient and periodically sample the GI tract directly.

Improving the AUC in an aqueous use environment means that amorphous Compound A may also provide enhanced bioavailability of Compound A by increasing the concentration of Compound A which remains dissolved in the use environment, particularly in the GI tract, and therefore increasing the amount of Compound A that is absorbed into the blood.

In another separate aspect, amorphous Compound A, when dosed orally to a human or other animal in the fasted state, provides improved concentration of dissolved Compound A in the blood relative to crystalline Compound A. An amorphous form of Compound A achieves a higher maximum drug concentration (Cₘₐₓ) of Compound A in the blood (serum or plasma) relative to a control composition consisting essentially of an equivalent amount of crystalline Form I of Compound A. It is to be understood that the crystalline Form I of Compound A is free from solubilizers or other components that would materially affect the solubility of Compound A in an appropriate use environment. For example, an amorphous form of Compound A provides a Cₘₐₓ of Compound A in the blood that is at least about 1.25-fold, or at least about 2-fold, or at least about 3-fold, that provided by the control composition consisting essentially of crystalline Form I of Compound A in an appropriate use environment.

In yet another aspect, an amorphous form of Compound A, when dosed orally to a human or other animal in the fasted state, provides an AUC of Compound A concentration in the blood (serum or plasma) that is at least about 1.25-fold, or at least about 2-fold, or at least about 3-fold that provided by the crystalline Form I of Compound A. It is noted that such compositions can also be said to have a relative bioavailability of from about 1.25-fold to about 3-fold that of the crystalline Form I of Compound A control.

The compositions of the present invention may comprise the amorphous Compound A alone, or may comprise excipients described in more detail below.

### Solid Amorphous Dispersions of Compound A and a Matrix

In another embodiment, the pharmaceutical composition comprises a solid amorphous dispersion of Compound A and one or more components, which are collectively referred to as the “matrix.” By “solid amorphous dispersion” is meant that at least a portion of Compound A is in the amorphous form and dispersed in the matrix. In a preferred embodiment, the matrix is selected such that the dispersion provides either improved physical stability, improved chemical stability, improved concentration-enhancement, or any combination of these or all three for Compound A as compared to undispersed amorphous Compound A alone. By “undispersed Compound A” is meant Compound A that is not dispersed in the matrix. The matrix may comprise a single component or it may be a mixture of two or more components. The components may be intimately mixed to form a single phase or molecular dispersion or they may exist as two or more distinct phases with differing compositions.

At least a portion of the matrix is either water swellable, dispersible, or soluble in aqueous solution at physiologically relevant pH (e.g., pH 1-8). The matrix as a whole should be a solid at room temperature, and remain substantially solid up to a temperature of at least about 40° C., preferably up to a temperature of at least about 60° C., and more preferably up to a temperature of at least about 70° C. In order to achieve this, the matrix should be comprised of at least one or more components with a melting point above about 40° C., preferably above about 60° C., and more preferably above about 70° C.

The amount of matrix relative to the amount of drug present in the dispersion of the present invention depends on the characteristics of the matrix and may vary widely from a drug-to-matrix weight ratio of from about 0.01 to about 100 (e.g., 1 wt % drug to 99 wt % drug). Preferably, Compound A-to-matrix weight ratio ranges from about 0.1 to about 49 (about 10 wt % drug to about 98 wt % drug).

The components used in the matrix may be polymeric or non-polymeric, and may comprise a mixture of several components. Thus the matrix may comprise a mixture of polymeric components, a mixture of non-polymeric components, or a mixture of polymeric and non-polymeric components.

The term “polymeric” is used conventionally, meaning a compound that is made of monomers connected together to form a larger molecule. Polymeric matrix components generally will result in dispersions with improved concentration enhancement relative to non-polymeric matrix components. The polymeric component may be neutral or ionizable, and may be cellulosic or non-cellulosic. Exemplary polymeric components for use as the matrix include concentration-enhancing polymers described herein below, polyethylene glycols, polyoxyethylene glycols, polyethylene oxides, xanthan gum, carrageenan, chitosan, polydextrose, dextrin and starch. Also included within this definition are high molecular weight proteins such as gelatin and albumin. Preferably, the matrix is a concentration-enhancing polymer, described below.

By “non-polymeric” is meant that the component is not polymeric. Exemplary non-polymeric materials for use as a matrix component include, but are not limited to: alcohols, such as stearyl alcohol and cetyl alcohol, organic acids and their salts, such as stearic acid, citric acid, fumaric acid, tartaric acid, malic acid, and pharmaceutically acceptable salts thereof; organic bases such as glucosamine, N-methylglucamine, tris (hydroxymethyl)amino methane, and dodecylamine; salts such as sodium chloride, potassium chloride, lithium chloride, calcium chloride, magnesium chloride, sodium sulfate, potassium sulfate, sodium carbonate, and magnesium sulfate; amino acids such as alanine and glycine; sugars such as glucose, sucrose, xylitol, fructose, lactose, trehalose, mannitol, sorbitol, and maltitol; fatty acid esters such as glyceryl (mono- and di-) stearates, glyceryl (mono- and di-) behenates, triglycerdes, sorbitan monostearate, saccharose monostearate, glyceryl (palmitic stearic) ester, hydrogenated cottonseed oil, polyoxyethylene sorbitan fatty-acid esters; waxes, such as microcrystalline wax, paraffin wax, beeswax, synthetic wax, castor wax, and carnauba wax; alkyl sulfates such as sodium lauryl sulfate and magnesium lauryl sulfate; and phospholipids, such as lecithin; and mixtures thereof.

In the compositions of the present invention, at least a major portion of Compound A present in the solid amorphous dispersion may be in an amorphous form. As used herein, the term “a major portion” of Compound A means that at least about 60 wt % of Compound A in the solid amorphous dispersion is in an amorphous form, rather than a crystalline form. Alternatively, the compositions of the present invention may comprise Compound A in a solid amorphous dispersion is that is substantially amorphous. As used herein, “substantially amorphous” means that the amount of Compound A in a crystalline form does not exceed about 25 wt %. Additionally the compositions of the present invention may comprise Compound A in the solid amorphous dispersion that is “almost completely amorphous,” meaning that the amount of Compound A in a crystalline form does not exceed about 10 wt % of the total amount of Compound A present. Amounts of crystalline Compound A may be determined by one of ordinary skill in the art using analytical techniques that include, but are not limited to, Powder X-Ray Diffraction (PXRD), Scanning Electron Microscope (SEM) analysis, differential scanning calorimetry (DSC), or any other standard quantitative measurement.

An amorphous form of Compound A can exist within the solid amorphous dispersion in relatively pure amorphous drug domains or regions, as a solid solution of drug homogeneously distributed throughout the polymer or any combination of these states or those states that lie intermediate between them.

### Compositions Comprising Concentration-Enhancing Polymers

In another embodiment, the pharmaceutical compositions comprise an amorphous form of Compound A and a concentration-enhancing polymer. The concentration-enhancing polymers are capable of further improving the concentration of dissolved drug in an appropriate use environment. In particular, an advantage of including a concentration-enhancing polymer in the compositions of the present invention is to improve the AUC of Compound A as compared to compositions comprising Compound A that do not include such a concentration-enhancing polymer. The inventors have found that amorphous Compound A when dissolved in an appropriate use environment provides an initial concentration of Compound A that exceeds the equilibrium concentration of Compound A; however, the concentration of dissolved Compound A decreases substantially over time. Without wishing to be bound by theory, the inventors believe that the addition of a concentration-enhancing polymer to the use environment with the dissolved Compound A retards the rate at which the initially enhanced dissolved drug concentration falls to the equilibrium concentration. The result is that the compositions comprising amorphous Compound A and a concentration-enhancing polymer provide an improved dissolution area-under-the-curve (“AUC”) that is greater than that provided by Compound A alone.

Improving the AUC means that the compositions comprising a concentration-enhancing polymer may also provide enhanced bioavailability of Compound A by increasing the concentration of drug which remains dissolved in a use environment, particularly in the gastrointestinal (GI) tract of a mammal, such as a human. Improving the concentration of Compound A in solution allows higher blood levels in a mammal to be achieved, in some cases enabling an effective level to be reached or in other cases, allowing effective blood levels to be reached at lower drug dosage levels, which in turn decreases the amount of drug that must be dosed, reduces the blood level variability, and also decreases the size of the dosage form, depending on the amount of polymer needed.

Concentration-enhancing polymers suitable for use in the compositions of the present invention should be inert, in the sense that they do not chemically react with Compound A in an adverse manner, and are pharmaceutically acceptable. The polymer can be neutral or ionizable, and should have an aqueous-solubility of at least about 0.1 mg/mL over at least a portion of the pH range of about 1-8. Concentration-enhancing polymers suitable for use with the present invention may be cellulosic or non-cellulosic. Of these, ionizable and cellulosic polymers are preferred, with ionizable cellulosic polymers being more preferred. By “cellulosic” is meant a cellulose polymer that has been modified by reaction of at least a portion of the hydroxyl groups on the saccharide repeating units with a compound to form an ester or an ether substituent.

A preferred class of polymers comprises polymers that are “amphiphilic” in nature, meaning that the polymer has hydrophobic and hydrophilic portions. The hydrophobic portion may comprise groups such as aliphatic or aromatic hydrocarbon groups. The hydrophilic portion may comprise either ionizable or non-ionizable groups that are capable of hydrogen bonding such as hydroxyls, carboxylic acids, esters, amines or amides.

One class of polymers suitable for use with the present invention comprises non-ionizable (or neutral) non-cellulosic polymers. Exemplary polymers include: vinyl polymers and copolymers having substituents of hydroxyl, alkylacyloxy, or cyclicamido; polyvinyl alcohols that have at least a portion of their repeat units in the unhydrolyzed (vinyl acetate) form; polyvinyl alcohol polyvinyl acetate copolymers; polyvinyl pyrrolidone; polyoxyethylene-polyoxypropylene copolymers, also known as poloxamers; and polyethylene polyvinyl alcohol copolymers. Exemplary non-cellulosic, neutral polymers include hydroxyethyl methacrylate, polyvinylhydroxyethyl ether, and polyethylene glycol.

A preferred class of neutral non-cellulosic polymers are comprised of vinyl copolymers of a hydrophilic, hydroxyl-containing repeat unit and a hydrophobic, alkyl- or aryl-containing repeat unit. Such neutral vinyl copolymers are termed “amphiphilic hydroxyl-functional vinyl copolymers.” Amphiphilic hydroxyl-functional vinyl copolymers are exceptional in that they are both non-ionic and yet, surprisingly, when used as dispersion polymers for low-solubility drugs, yield solid amorphous dispersions that provide high levels of drug concentration enhancement when dosed to an aqueous environment of use.

The preferred copolymers have the general structure: 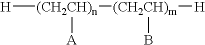
where A and B represent “hydrophilic, hydroxyl-containing” and “hydrophobic” substituents, respectively, and n and m represent the average number of hydrophilic vinyl repeat units and average number of hydrophobic vinyl repeat units respectively per polymer molecule. Copolymers may be block copolymers, random copolymers or they may have structures anywhere between these two extremes. The polymers may have, for example, molecular weights from about 2,500 to about 1,000,000 daltons.

The hydrophilic, hydroxyl-containing repeat units, “A,” may simply be hydroxyl (—OH) or it may be any short-chain, 1 to 6 carbon, alkyl with one or more hydroxyls attached thereto. The hydroxyl-substituted alkyl may be attached to the vinyl backbone via carbon-carbon or ether linkages. Thus, exemplary “A” structures include, in addition to hydroxyl itself, hydroxymethyl, hydroxyethyl, hydroxypropyl, hydroxymethoxy, hydroxyethoxy and hydroxypropoxy.

The hydrophobic substituent, “B,” may simply be: hydrogen (—H), in which case the hydrophobic repeat unit is ethylene; an alkyl or aryl substituent with up to 12 carbons attached via a carbon-carbon bond such as methyl, ethyl or phenyl; an alkyl or aryl substituent with up to 12 carbons attached via an ether linkage such as methoxy, ethoxy or phenoxy; an alkyl or aryl substituent with up to 12 carbons attached via an ester linkage such as acetate, propionate, butyrate or benzoate. The amphiphilic hydroxyl-functional vinyl copolymers of the present invention may be synthesized by any conventional method used to prepare substituted vinyl copolymers. Some substituted vinyl copolymers such as polyvinyl alcohol/polyvinyl acetate are well known and commercially available.

Such polymers are more fully disclosed in commonly assigned pending U.S. patent application Ser. No. 10/175,132, which claims priority to provisional application Ser. No. 60/300,255, filed Jun. 22, 2001, herein incorporated by reference.

Another class of polymers suitable for use with the present invention comprises ionizable non-cellulosic polymers. Exemplary polymers include: carboxylic acid-functionalized vinyl polymers, such as the carboxylic acid functionalized polymethacrylates and carboxylic acid functionalized polyacrylates such as the EUDRAGITS® manufactured by Rohm Tech Inc., of Malden, Mass.; amine-functionalized polyacrylates and polymethacrylates; proteins; and carboxylic acid functionalized starches such as starch glycolate.

Non-cellulosic polymers that are amphiphilic are copolymers of a relatively hydrophilic and a relatively hydrophobic monomer. Examples include acrylate and methacrylate copolymers, and polyoxyethylene-polyoxypropylene copolymers. Exemplary commercial grades of such copolymers include the EUDRAGITS, which are copolymers of methacrylates and acrylates, and the PLURONICS supplied by BASF, which are polyoxyethylene-polyoxypropylene copolymers.

A preferred class of polymers comprises ionizable and neutral cellulosic polymers with at least one ester- and/or ether-linked substituent in which the polymer has a degree of substitution of at least 0.1 for each substituent. It should be noted that in the polymer nomenclature used herein, ether-linked substituents are recited prior to “cellulose” as the moiety attached to the ether group; for example, “ethylbenzoic acid cellulose” has ethoxybenzoic acid substituents. Analogously, ester-linked substituents are recited after “cellulose” as the carboxylate; for example, “cellulose phthalate” has one carboxylic acid of each phthalate moiety ester-linked to the polymer and the other carboxylic acid unreacted.

It should also be noted that a polymer name such as “cellulose acetate phthalate” (CAP) refers to any of the family of cellulosic polymers that have acetate and phthalate groups attached via ester linkages to a significant fraction of the cellulosic polymer's hydroxyl groups. Generally, the degree of substitution of each substituent group can range from 0.1 to 2.9 as long as the other criteria of the polymer are met. “Degree of substitution” refers to the average number of the three hydroxyls per saccharide repeat unit on the cellulose chain that have been substituted. For example, if all of the hydroxyls on the cellulose chain have been phthalate substituted, the phthalate degree of substitution is 3. Also included within each polymer family type are cellulosic polymers that have additional substituents added in relatively small amounts that do not substantially alter the performance of the polymer. Thus, for example, the polymer name “hydroxypropyl methyl cellulose acetate succinate” includes the commercial grades L, M, and H available from Shin Etsu, Tokyo, Japan.

Amphiphilic cellulosics comprise polymers in which the parent cellulosic polymer has been substituted at any or all of the 3 hydroxyl groups present on each saccharide repeat unit with at least one relatively hydrophobic substituent. Hydrophobic substituents may be essentially any substituent that, if substituted to a high enough level or degree of substitution, can render the cellulosic polymer essentially aqueous insoluble. Examples of hydrophobic substituents include ether-linked alkyl groups such as methyl, ethyl, propyl, butyl, etc.; or ester-linked alkyl groups such as acetate, propionate, butyrate, etc.; and ether- and/or ester-linked aryl groups such as phenyl, benzoate, or phenylate. Hydrophilic regions of the polymer can be either those portions that are relatively unsubstituted, since the unsubstituted hydroxyls are themselves relatively hydrophilic, or those regions that are substituted with hydrophilic substituents. Hydrophilic substituents include ether- or ester-linked nonionizable groups such as the hydroxy alkyl substituents hydroxyethyl, hydroxypropyl, and the alkyl ether groups such as ethoxyethoxy or methoxyethoxy. Particularly preferred hydrophilic substituents are those that are ether- or ester-linked ionizable groups such as carboxylic acids, thiocarboxylic acids, substituted phenoxy groups, amines, phosphates or sulfonates.

One class of cellulosic polymers comprises neutral polymers, meaning that the polymers are substantially non-ionizable in aqueous solution. Such polymers contain non-ionizable substituents, which may be either ether-linked or ester-linked. Exemplary ether-linked non-ionizable substituents include: alkyl groups, such as methyl, ethyl, propyl, butyl, etc.; hydroxy alkyl groups such as hydroxymethyl, hydroxyethyl, hydroxypropyl, etc.; and aryl groups such as phenyl. Exemplary ester-linked non-ionizable substituents include: alkyl groups, such as acetate, propionate, butyrate, etc.; and aryl groups such as phenylate. However, when aryl groups are included, the polymer may need to include a sufficient amount of a hydrophilic substituent so that the polymer has at least some water solubility at any physiologically relevant pH of from 1 to 8.

Exemplary non-ionizable polymers that may be used as the polymer include: hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

A preferred set of neutral cellulosic polymers are those that are amphiphilic. Exemplary polymers include hydroxypropyl methyl cellulose and hydroxypropyl cellulose acetate, where cellulosic repeat units that have relatively high numbers of methyl or acetate substituents relative to the unsubstituted hydroxyl or hydroxypropyl substituents constitute hydrophobic regions relative to other repeat units on the polymer.

A preferred class of cellulosic polymers comprises polymers that are at least partially ionizable at physiologically relevant pH and include at least one ionizable substituent, which may be either ether-linked or ester-linked. Exemplary ether-linked ionizable substituents include: carboxylic acids, such as acetic acid, propionic acid, benzoic acid, salicylic acid, alkoxybenzoic acids such as ethoxybenzoic acid or propoxybenzoic acid, the various isomers of alkoxyphthalic acid such as ethoxyphthalic acid and ethoxyisophthalic acid, the various isomers of alkoxynicotinic acid such as ethoxynicotinic acid, and the various isomers of picolinic acid such as ethoxypicolinic acid, etc.; thiocarboxylic acids, such as thioacetic acid; substituted phenoxy groups, such as hydroxyphenoxy, etc.; amines, such as aminoethoxy, diethylaminoethoxy, trimethylaminoethoxy, etc.; phosphates, such as phosphate ethoxy; and sulfonates, such as sulphonate ethoxy. Exemplary ester linked ionizable substituents include: carboxylic acids, such as succinate, citrate, phthalate, terephthalate, isophthalate, trimellitate, and the various isomers of pyridinedicarboxylic acid, etc.; thiocarboxylic acids, such as thiosuccinate; substituted phenoxy groups, such as amino salicylic acid; amines, such as natural or synthetic amino acids, such as alanine or phenylalanine; phosphates, such as acetyl phosphate; and sulfonates, such as acetyl sulfonate. For aromatic-substituted polymers to also have the requisite aqueous solubility, it is also desirable that sufficient hydrophilic groups such as hydroxypropyl or carboxylic acid functional groups be attached to the polymer to render the polymer aqueous soluble at least at pH values where any ionizable groups are ionized. In some cases, the aromatic group may itself be ionizable, such as phthalate or trimellitate substituents.

Exemplary cellulosic polymers that are at least partially ionized at physiologically relevant pHs include: hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, carboxymethylethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Exemplary ionizable cellulosic polymers that meet the definition of amphiphilic, having hydrophilic and hydrophobic regions, include polymers such as cellulose acetate phthalate and cellulose acetate trimellitate where the cellulosic repeat units that have one or more acetate substituents are hydrophobic relative to those that have no acetate substituents or have one or more ionized phthalate or trimellitate substituents.

A particularly desirable subset of cellulosic ionizable polymers are those that possess both a carboxylic acid functional aromatic substituent and an alkylate substituent and thus are amphiphilic. Exemplary polymers include cellulose acetate phthalate, methyl cellulose acetate phthalate, ethyl cellulose acetate phthalate, hydroxypropyl cellulose acetate phthalate, hydroxypropyl methyl cellulose phthalate, hydroxypropyl methyl cellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Another particularly desirable subset of cellulosic ionizable polymers are those that are amphiphilic and possess a non-aromatic carboxylate substituent. Exemplary polymers include hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, hydroxyethyl cellulose acetate succinate, and carboxymethyl ethyl cellulose.

Another preferred class of polymers consists of neutralized acidic polymers. By “neutralized acidic polymer” is meant any acidic polymer for which a significant fraction of the “acidic moieties” or “acidic substituents” have been “neutralized”; that is, exist in their deprotonated form. By “acidic polymer” is meant any polymer that possesses a significant number of acidic moieties. In general, a significant number of acidic moieties would be greater than or equal to about 0.1 milliequivalents of acidic moieties per gram of polymer. “Acidic moieties” include any functional groups that are sufficiently acidic that, in contact with or dissolved in water, can at least partially donate a hydrogen cation to water and thus increase the hydrogen-ion concentration. This definition includes any functional group or “substituent,” as it is termed when the functional group is covalently attached to a polymer, that has a pKₐ of less than about 10. Exemplary classes of functional groups that are included in the above description include carboxylic acids, thiocarboxylic acids, phosphates, phenylic groups, and sulfonates. Such functional groups may make up the primary structure of the polymer such as for polyacrylic acid, but more generally are covalently attached to the backbone of the parent polymer and thus are termed “substituents.”

The “degree of neutralization,” a, of a polymer substituted with monoprotic acids (such as carboxylic acids) is defined as the fraction of the acidic moieties on the polymer that have been neutralized; that is, deprotonated by a base. Typically, for an acidic polymer to be considered a “neutralized acidic polymer,” α must be at least about 0.001 (or 0.1%), preferably about 0.01 (1%) and more preferably at least about 0.1 (10%). Such small degrees of neutralization may be acceptable because often the effective pH of the polymer changes dramatically with small increases in the degree of neutralization. Nonetheless, even greater degrees of neutralization are even more preferred. Thus, a is preferably at least 0.5 (meaning that at least 50% of the acidic moieties have been neutralized) and α is more preferably at least 0.9 (meaning that at least 90% of the acidic moieties have been neutralized).

Neutralized acidic polymers are described in more detail in commonly assigned pending U.S. patent application Ser. No. 10/175,566, which claims priority from U.S. provisional patent application Ser. No. 60/300,256 entitled “Pharmaceutical Compositions of Drugs and Neutralized Acidic Polymers” filed Jun. 22, 2001, the relevant disclosures of which is incorporated by reference.

While specific polymers have been discussed as being suitable for use in the compositions of the present invention, blends of such polymers may also be suitable. Thus the term “polymer” is intended to include blends of polymers in addition to a single species of polymer.

The amount of concentration-enhancing polymer present in the composition is sufficient to provide concentration-enhancement, as described in more detail below. In general, the ratio of drug to polymer may range from about 0.01 (1 part drug to 100 parts polymer) to about 100.

The amorphous Compound A and concentration-enhancing polymer may be combined in any manner. In one embodiment, the composition comprises a combination of amorphous Compound A and the concentration-enhancing polymer. In another embodiment, the composition comprises a combination of (1) a solid amorphous dispersion comprising Compound A and a matrix and (2) the concentration-enhancing polymer. “Combination” as used herein means that the amorphous Compound A or the solid amorphous dispersion comprising Compound A and a matrix and the concentration-enhancing polymer may be in physical contact with each other or in close proximity but without the necessity of being physically mixed. For example, the composition may be in the form of a multi-layer tablet, as known in the art, wherein one or more layers comprises amorphous Compound A and one or more different layers comprises the concentration-enhancing polymer. Yet another example may constitute a coated tablet wherein either Compound A or the concentration-enhancing polymer or both may be present in the tablet core and the coating may comprise amorphous Compound A or the concentration-enhancing polymer or both. Alternatively, the combination can be in the form of a simple dry physical mixture wherein both the amorphous Compound A and concentration-enhancing polymer are mixed in particulate form and wherein the particles of each, regardless of size, retain the same individual physical properties that they exhibit in bulk.

Combinations of amorphous Compound A and concentration-enhancing polymer may be formed in any conventional way such as by blending the dry ingredients including the amorphous Compound A, one or more concentration-enhancing polymers, and any other excipients appropriate to forming the desired dosage form using V-blenders, planetary mixers, vortex blenders, mills, extruders such as twin-screen extruders and trituration processes. The ingredients can be combined in granulation processes utilizing mechanical energy, such as ball mills or roller compactors. They may also be combined using wet granulation methods in high-shear granulators or fluid bed granulators wherein a solvent or wetting agent is added to the ingredients or the concentration-enhancing polymer may be dissolved in a solvent and used as a granulating fluid. The concentration-enhancing polymer may be added as a coating to tablets preformed by a compression process from a mixture containing amorphous Compound A, the coating taking place in a spray-coating process using, for example, a pan coater or a fluidized-bed coater.

Alternatively, the compositions of the present invention may be co-administered, meaning that the amorphous drug can be administered separately from, but within the same general time frame as, the concentration-enhancing polymer. Thus, the amorphous drug can, for example, be administered in its own dosage form, which is taken at approximately the same time as the concentration-enhancing polymer that is in a separate dosage form. If administered separately, it is generally preferred to administer both the amorphous Compound A and the concentration-enhancing polymer within 60 minutes of each other, so that the two are present together in the environment of use. When not administered simultaneously, the concentration-enhancing polymer is preferably administered prior to the amorphous form of Compound A.

In another embodiment, Compound A and the concentration-enhancing polymer are combined and formed into a solid amorphous dispersion. Solid amorphous dispersions comprising Compound A and concentration-enhancing polymers are preferred because such solid amorphous dispersions are often capable of achieving high concentrations of dissolved drug in in vitro and in vivo use environments.

The solid amorphous dispersions comprising concentration-enhancing polymers may contain up to about 99 wt % Compound A, depending on the dose of Compound A and the effectiveness of the concentration-enhancing polymer. Amorphous dispersions may be formed containing very high loadings of Compound A. When the solid amorphous dispersion consists only of Compound A and concentration-enhancing polymer, the solid amorphous dispersion has a drug to polymer weight ratio of at least about 0.67. The solid amorphous dispersion may comprise at least about 30 wt %, at least about 40 wt %, at least about 50 wt %, at least about 60 wt %, at least about 70 wt %, at least about 80 wt %, at least about 90 wt %, or at least about 95 wt % Compound A, or a pharmaceutically acceptable salt or solvate thereof. Solid amorphous dispersions comprising Compound A in a matrix, such as a matrix comprising cellulosic polymers such as HPMCAS (hydroxypropyl methyl cellulose acetate succinate), can be physically stable over extended periods of time, in that the dispersions continue to provide improved dissolution performance of Compound A even after storage for extended periods of time.

### Stabilizing Agents

In one embodiment, the composition further comprises a stabilizing agent to promote the chemical stability of Compound A. The stabilizing agent reduces the rate at which Compound A degrades into another chemical compound or compounds through, for example, oxidative degradation processes. To prevent degradation, the compositions of the present invention may include a stabilizing agent. One class of useful stabilizing agents is an anti-oxidants. Exemplary anti-oxidants that may be included in the dispersion include, but are not limited to, butylated hydroxytoluene (BHT), butylated hydroxyanisole (BHA), propyl gallate, vitamin E, and vitamin E succinate. Such anti-oxidants may be present in amounts sufficient to reduce oxidation, but not exceeding pharmaceutically acceptable amounts. Exemplary amounts range from about 0 to about 10 wt %.

Another stabilizing agent that is useful to include is a base. Examples of bases include (but are not limited to) hydroxides, such as sodium hydroxide, calcium hydroxide, ammonium hydroxide, and choline hydroxide; bicarbonates, such as (but not limited to) sodium bicarbonate, potassium bicarbonate, and ammonium bicarbonate; carbonates, such as (but not limited to) ammonium carbonate, calcium carbonate, and sodium carbonate; amines, such as (but not limited to) tris(hydroxymethyl)amino methane, ethanolamine, diethanolamine, N-methyl glucamine, glucosamine, ethylenediamine, N,N′-dibenzylethylenediamine, N-benzyl-2-phenethylamine, cyclohexylamine, cyclopentylamine, diethylamine, isopropylamine, diisopropylamine, dodecylamine, and triethylamine; proteins, such as (but not limited to) gelatin; amino acids such as (but not limited to) lysine, arginine, guanine, glycine, and adenine; polymeric amines, such as polyamino methacrylates, such as Eudragit E; conjugate bases of various acids, such as (but not limited to) sodium acetate, potassium acetate, calcium acetate, magnesium acetate, ammonium acetate, potassium citrate, calcium citrate, sodium citrate, disodium citrate, trisodium citrate, sodium benzoate, potassium benzoate, calcium benzoate, sodium propionate, disodium phosphate, trisodium phosphate, calcium hydrogen phosphate, sodium phenylate, sodium sulfate, ammonium chloride, and ammonium sulfate; salts of EDTA (ethylenediaminetetraacetic acid), such as (but not limited to) tetra sodium EDTA; salts of various acidic polymers such as sodium starch glycolate, sodium carboxymethyl cellulose and sodium polyacrylic acid; and N-methylmorpholine. Preferred bases are conjugate bases of organic acids, such as (but not limited to) sodium acetate, potassium acetate, calcium acetate, magnesium acetate, ammonium acetate, potassium citrate, calcium citrate, sodium citrate, disodium citrate, trisodium citrate, sodium benzoate, potassium benzoate, calcium benzoate, and sodium propionate.

Addition of base to a solid amorphous dispersion is particularly preferred where the dispersion polymer is acidic, such as HPMCAS. In this embodiment, the base may be present in a sufficient amount to neutralize a significant amount of the acidic groups on the polymer. The base may be present in a sufficient amount to neutralize at least about 40% of the acidic groups, at least about 50% of the acidic groups, or at least about 90% of the acidic groups. The base may even be present in excess of the acidic groups so that essentially 100% of the acidic groups are neutralized.

When the neutralized form of the acidic polymer comprises a multivalent cationic species such as Ca²⁺, Mg²⁺, Al³⁺, Fe²⁺, Fe³⁺, or a diamine, such as ethylene diamine, the cationic species may interact with two or more neutralized acidic moieties on more than one polymer chain, resulting in an ionic crosslink between the polymer chains. An acidic polymer may be considered “ionically crosslinked” if the number of milliequivalents of multivalent cationic species per gram of polymer is at least about 5%, preferably at least about 10%, the number of milliequivalents of acidic moieties (of the polymer) per gram of polymer. Alternatively, an acidic polymer may be considered “ionically crosslinked” if sufficient multivalent cationic species are present such that the neutralized acidic polymer has a higher glass transition temperature (T_{g}) than the same polymer containing essentially no multivalent cationic species. Mobility in dispersions formed from such ionically crosslinked polymers is particularly low relative to dispersions formed from the acidic form of the same polymers. Such ionically crosslinked polymers may be formed by neutralization of the acidic polymer using any base where the cationic counterion of the base is divalent. Thus, magnesium hydroxide, calcium acetate or ethylene diamine may be added to an acidic polymer such as cellulosic acetate phthalate or hydroxypropyl methyl cellulose acetate succinate to form a neutralized, ionically crosslinked, acidic cellulosic polymer. Low mobility in such polymers may be indicated by high T_{g} values or, more typically, a decrease in the magnitude of the heat capacity increase in the vicinity of the T_{g} or, in some cases, the absence of any apparent T_{g} when the dispersion is subjected to differential thermal analysis. Thus, when the polymer is essentially completely neutralized, no T_{g} is apparent when the neutralized polymer is subjected to differential thermal analysis. Such ionically cross-linked polymers may provide improved physical or chemical stability for Compound A in the dispersion relative to non-ionically crosslinked neutralized acidic polymer.

### Preparation of Amorphous Compound A

Amorphous Compound A and solid amorphous dispersions of Compound A may be made according to any conventional process known to those of ordinary skill in the art. Such processes include mechanical, thermal and solvent processes. Exemplary mechanical processes include milling and extrusion; melt processes including high temperature fusion, solvent-modified fusion and melt-congeal processes; and solvent processes including non-solvent precipitation, spray coating and spray drying. Often, processes may form the dispersion by a combination of two or more process types. For example, when an extrusion process is used the extruder may be operated at an elevated temperature such that both mechanical (shear) and thermal (heat) means are used to form the dispersion. Examples of exemplary methods are disclosed in the following U.S. Patents, the pertinent disclosures of which are incorporated herein by reference: Nos. 5,456,923 and 5,939,099, which describe forming dispersions by extrusion processes; Nos. 5,340,591 and 4,673,564, which describe forming dispersions by milling processes; and Nos. 5,707,646 and 4,894,235, which describe forming dispersions by melt congeal processes.

In one embodiment, the dispersion is formed by a thermal process, such as an extrusion process, a fusion process, or a melt-congeal process. In such cases, the matrix is selected such that it is suitable for use in the thermal process. Generally, it is desirable to keep the processing temperature as low as possible to avoid thermal degradation of Compound A. As such, it is preferred that the matrix as a whole become fluid at a temperature of less than about 200° C., more preferably less than about 160° C., and most preferably less than about 120° C.

Exemplary materials that are suitable for use as a matrix component for thermal processes include: alcohols, such as stearyl alcohol and cetyl alcohol, organic acids and pharmaceutically acceptable salts thereof, such as stearic acid, citric acid, and malic acid; sugars such as glucose, trehalose, xylitol, sorbitol, and maltitol; fatty acid esters such as mono-, di-, and tri-glycerides, glyceryl mono-, di-, and tri-stearates, glyceryl mono-, di-, and tri-behenates, sorbitan monostearate, saccharose monostearate, glyceryl (palmitic stearic) ester, hydrogenated cottonseed oil, polyoxyethylene sorbitan fatty-acid esters; waxes, such as microcrystalline wax, paraffin wax, beeswax, synthetic wax, castor wax, and carnauba wax; alkyl sulfates such as sodium lauryl sulfate; and polymers such as polyethylene glycols, polyoxyethylene glycols, polyethylene-propylene glycol copolymers, poloxamers, polyethylene oxide, polyvinyl pyrrolidinone (also referred to as polyvinyl pyrrolidone or povidone or PVP), polyvinyl alcohol, polyethylene-vinyl alcohol copolymers, polyvinyl alcohol polyvinyl acetate copolymers, carboxylic acid-functionalized polymethacrylates, and amine-functionalized polymethacrylates. While specific materials have been discussed as being suitable for use alone in the dispersions formed by thermal processing, blends of materials may also be suitable. For example, a water-insoluble matrix component such as microcrystalline wax may be blended with a highly water soluble matrix component, such as a poloxamer, to form a water-dispersible matrix.

The matrix may include a plasticizer as one component of the matrix to reduce processing temperature. Exemplary plasticizers include mineral oils, petrolatum, lanolin alcohols, polyethylene glycol, polypropylene glycol, sorbitol, triethanol amine, benzyl benzoate, dibutyl sebacate, diethyl phthalate, glyceryl monostearate, triacetin, and triethyl citrate. The amount of plasticizer used will depend on the melting point of the other matrix components and the desired processing temperature. Typically, the ratio of plasticizer to matrix will be 0.01 to 0.5, more typically 0.05 to 0.1. Solvents or swelling agents, such as water, alcohols, ketones, and the like may also be used to reduce processing temperature and improve the processability of the composition.

One preferred thermal process is an extrusion process. Here, Compound A and the one or more matrix components may be dry blended, with or without the addition of a plasticizer, and the blend fed to a twin-screw extrusion device. The twin-screw extrusion device is designed such that there is sufficient heat and mechanical energy (e.g., shear) to form a dispersion, without degradation of Compound A or matrix. The processing temperature may vary from about 50° C. up to about 200° C., depending on the melting point of the matrix materials. Generally, the higher the melting point of the matrix components, the higher the processing temperature.

When Compound A has a high solubility in the matrix, a lower amount of mechanical energy will be required to form the dispersion. In such cases, the processing temperature may be below the melting temperature of Compound A but greater than the melting point of at least a portion of the matrix materials, since Compound A will dissolve into the molten matrix.

When Compound A has a low solubility in the matrix, a higher amount of mechanical energy may be required to form the dispersion. Here, the processing temperature may need to be above the melting point of Compound A and at least some of the matrix components. A high amount of mechanical energy may be needed to mix the molten Compound A with the matrix components to form a dispersion. Typically, the lowest processing temperature and an extruder design that imparts the lowest amount of mechanical energy (e.g., shear) that produce a satisfactory dispersion is chosen in order to minimize the exposure of Compound A to harsh conditions.

Another preferred method for forming dispersions is “solvent processing,” which consists of dissolution of at least a portion of Compound A and at least a portion of the one or more matrix components in a common solvent. The term “solvent” is used broadly and includes mixtures of solvents. “Common” here means that the solvent, which can be a mixture of compounds, will dissolve at least a portion of Compound A and the matrix material(s).

Exemplary materials that are suitable for use as a matrix component for solvent processing include the concentration-enhancing polymers previously described; alcohols, such as stearyl alcohol and cetyl alcohol; organic acids and their pharmaceutically acceptable salts, such as stearic acid, citric acid, fumaric acid, tartaric acid, and malic acid; salts such as sodium chloride, potassium chloride, lithium chloride, calcium chloride, magnesium chloride, sodium sulfate, potassium sulfate, sodium carbonate, and magnesium sulfate; amino acids such as alanine and glycine; sugars such as glucose, sucrose, xylitol, fructose, lactose, mannitol, sorbitol, and maltitol; fatty acid esters such as glyceryl (mono- and di-) stearates, triglycerides, hydrogenated cottonseed oil, sorbitan monostearate, saccharose monostearate, glyceryl (palmitic stearic) ester, polyoxyethylene sorbitan fatty-acid esters; waxes, such as microcrystalline wax, paraffin wax, beeswax, synthetic wax, castor wax, and carnauba wax; alkyl sulfates such as sodium lauryl sulfate and magnesium lauryl sulfate; phospholipids, such as lecithin; proteins, such as gelatin and albumin; and polymers such as polyethylene glycols, polyoxyethylene glycols, polyethylene oxides, xanthan gum, carrageenan, chitosan, polydextrose, dextrin, and starch. While specific materials have been discussed as being suitable for use alone in the dispersions formed by solvent processing, blends of materials may also be suitable.

After at least a portion of Compound A and matrix have been dissolved, the solvent is removed by evaporation or by mixing with a non-solvent. Exemplary processes are spray-drying, spray-coating (pan-coating, fluidized bed coating, etc.), and precipitation by rapid mixing of the Compound A and matrix solution with CO₂, hexane, heptane, water of appropriate pH, or some other non-solvent. Preferably, removal of the solvent results in a solid dispersion that is substantially homogeneous. To achieve this end, it is generally desirable to rapidly remove the solvent from the solution such as in a process where the solution is atomized and the Compound A and matrix rapidly solidify.

After Compound A and the matrix have been dissolved, the solvent is rapidly removed by evaporation or by mixing with a non-solvent. Exemplary processes are spray-drying, spray-coating (pan-coating, fluidized bed coating, etc.), and precipitation by rapid mixing of the matrix and Compound A solution with CO₂, water, or some other non-solvent.

In the case of solid amorphous dispersions, the dispersion may be phase separated, meaning Compound A and matrix are each in separate domains within the dispersion as described above, or may be homogeneously distributed throughout each other to form a single phase, or any combination of these or those states that lie intermediate. Preferably, removal of the solvent results in the formation of a substantially homogeneous, solid amorphous dispersion. In such dispersions, Compound A and the matrix are dispersed as homogeneously as possible throughout each other and can be thought of as a solid solution of Compound A dispersed in the matrix, wherein the solid amorphous dispersion is thermodynamically stable, meaning that the concentration of Compound A in the matrix is at or below its equilibrium value, or it may be considered to be a supersaturated solid solution where Compound A concentration in the matrix is above its equilibrium value.

The solvent may be removed by spray-drying. The term “spray-drying” is used conventionally and broadly refers to processes involving breaking up liquid mixtures into small droplets (atomization) and rapidly removing solvent from the mixture in a spray-drying apparatus where there is a strong driving force for evaporation of solvent from the droplets. Spray-drying processes and spray-drying equipment are described generally in Perry's Chemical Engineers' Handbook, pages 20-54 to 20-57 (Sixth Edition 1984). More details on spray-drying processes and equipment are reviewed by Marshall, “Atomization and Spray-Drying,” 50 Chem. Eng. Prog. Monogr. Series 2 (1954), and Masters, Spray Drying Handbook (Fourth Edition 1985). The strong driving force for solvent evaporation is generally provided by maintaining the partial pressure of solvent in the spray-drying apparatus well below the vapor pressure of the solvent at the temperature of the drying droplets. This is accomplished by (1) maintaining the pressure in the spray-drying apparatus at a partial vacuum (e.g., 0.01 to 0.50 atm); or (2) mixing the liquid droplets with a warm drying gas; or (3) both (1) and (2). In addition, at least a portion of the heat required for evaporation of solvent may be provided by heating the spray solution.

Solvents suitable for spray-drying can be any compound in which Compound A and the matrix are mutually soluble. Preferably, the solvent is also volatile with a boiling point of 150° C. or less. In addition, the solvent should have relatively low toxicity and be removed from the solid amorphous dispersion to a level that is acceptable according to The International Committee on Harmonization (ICH) guidelines. Removal of solvent to this level may require a subsequent processing step such as tray-drying. Suitable solvents include, but are not limited to, alcohols such as methanol, ethanol, n-propanol, iso-propanol, and butanol; ketones such as acetone, methyl ethyl ketone and methyl iso-butyl ketone; esters such as ethyl acetate and propylacetate; and various other solvents such as acetonitrile, methylene chloride, toluene, and 1,1,1-trichloroethane. Lower volatility solvents such as dimethyl acetamide or dimethylsulfoxide can also be used. Mixtures of solvents, such as 50% methanol and 50% acetone, can also be used, as can mixtures with water, so long as the polymer and Compound A are sufficiently soluble to make the spray-drying process practicable.

Addition of water to the spray solution may improve the chemical stability of the resulting amorphous Compound A. In general, water may be present in an amount up to about 30 wt % of the solvent, depending on the concentration-enhancing polymer and other solvents present. In one embodiment, the solvent comprises methanol and water in a ratio of about 80/20 (wt/wt).

Another method for improving the chemical stability of the resulting amorphous Compound A is to purge the spray solution of oxygen by bubbling an inert gas such as nitrogen through the spray solution.

When the solid amorphous dispersion comprises a base, care should be taken to ensure that the base does not degrade Compound A. Compound A is susceptible to hydrolysis in the presence of a strong base. Therefore, where a base is used to neutralize an acidic polymer, the base and acidic polymer are preferably first combined in the solvent so that the base first reacts with the polymer. Compound A is then added to form the spray solution.

In addition, when base is added to the spray solution to neutralize an acidic polymer, it may be necessary to add water to the spray solution to provide sufficient solubility of the polymer in the spray solution. The spray solution may comprise up to 30 wt % water. For example, to achieve a spray solution having 10 wt % solids (comprising 90% Compound A, 8% HPMCAS, and 2% calcium acetate), the spray solution may comprise 80% methanol and 20% water (by weight).

The amount of Compound A and matrix in the spray solution depends on the solubility of each in the spray solution and the desired ratio of Compound A to matrix in the resulting solid amorphous dispersion. Preferably, the spray solution comprises at least about 1 wt %, more preferably at least about 3 wt %, and even more preferably at least about 10 wt % dissolved solids. Compound A to matrix ratio may range from 0.01 up to 100. Preferably, Compound A to matrix ratio is from at least about 0.66 to about 49, and more preferably from about 3 to about 19, and even more preferably from about 5 to about 15.

The solvent-bearing feed can be spray-dried under a wide variety of conditions and yet still yield amorphous drug or solid amorphous dispersions with acceptable properties. For example, various types of nozzles can be used to atomize the spray solution, thereby introducing the spray solution into the spray-dry chamber as a collection of small droplets. Essentially any type of nozzle may be used to spray the solution as long as the droplets that are formed are sufficiently small that they dry sufficiently (due to evaporation of solvent) such that they do not stick to or coat the spray-drying chamber wall.

Although the maximum droplet size varies widely as a function of the size, shape and flow pattern within the spray-dryer, generally droplets should be less than about 500 μm in diameter when they exit the nozzle. Examples of types of nozzles that may be used to form the solid amorphous dispersions include the two-fluid nozzle, the fountain-type nozzle, the flat fan-type nozzle, the pressure nozzle and the rotary atomizer. In a preferred embodiment, a pressure nozzle is used, as disclosed in detail in commonly assigned copending U.S. patent application Ser. No. 10/351,568, filed Jan. 24, 2003, which claimed priority to U.S. Provisional Application No. 60/353,986, filed Feb. 1, 2002, the disclosure of which is incorporated herein by reference.

The spray solution can be delivered to the spray nozzle or nozzles at a wide range of temperatures and flow rates. Generally, the spray solution temperature can range anywhere from just above the solvent's freezing point to about 20° C. above its ambient pressure boiling point (by pressurizing the solution) and in some cases even higher. Spray solution flow rates to the spray nozzle can vary over a wide range depending on the type of nozzle, spray-dryer size and spray-dry conditions such as the inlet temperature and flow rate of the drying gas. Generally, the energy for evaporation of solvent from the spray solution in a spray-drying process comes primarily from the drying gas.

The drying gas, in principle, can be essentially any gas, but for safety reasons and to minimize undesirable oxidation of Compound A or other materials in the solid amorphous dispersion, an inert gas such as nitrogen, nitrogen-enriched air or argon may be utilized. The drying gas is typically introduced into the drying chamber at a temperature between about 60° and about 300° C. and preferably between about 80° and about 240° C. In order to minimize chemical degradation of Compound A during and after the spray drying process, it is preferable to limit exposure of Compound A to high temperatures. For example, where the spray solution comprises Compound A, HPMCAS, methanol and water, and is sprayed using a NIRO PSD-1 Spray drier, the inlet gas temperature may be 150° C. or less, and more preferably 130° C. or less.

The large surface-to-volume ratio of the droplets and the large driving force for evaporation of solvent leads to rapid solidification times for the droplets. Solidification times should be less than about 20 seconds, preferably less than about 10 seconds, and more preferably less than 1 second. This rapid solidification is often critical to the particles maintaining a uniform, homogeneous dispersion instead of separating into Compound A-rich and polymer-rich phases. In a preferred embodiment, the height and volume of the spray-dryer are adjusted to provide sufficient time for the droplets to dry prior to impinging on an internal surface of the spray-dryer, as described in detail in commonly assigned U.S. Pat. No. 6,763,607, incorporated herein by reference. As noted above, to obtain large enhancements in concentration and bioavailability it is often necessary to obtain as homogeneous a dispersion as possible.

Following solidification, the solid powder typically stays in the spray-drying chamber for about 5 to 60 seconds, further evaporating solvent from the solid powder. The final solvent content of the solid dispersion as it exits the dryer should be low, since this reduces the mobility of Compound A molecules in the solid amorphous dispersion, thereby improving its stability. Generally, the solvent content of the solid amorphous dispersion as it leaves the spray-drying chamber should be less than 10 wt % and preferably less than 2 wt %.

Following formation, the solid amorphous dispersion can be dried to remove residual solvent using suitable drying processes, such as tray drying, vacuum drying, fluid bed drying, microwave drying, belt drying, rotary drying, and other drying processes known in the art. Preferably, the solid amorphous dispersions are dried under conditions that minimize exposure to hot, dry conditions. Preferably, the temperature is less than 50° C., and more preferably less than 40° C. The relative humidity is preferably maintained between 25% and 75% relative humidity. Preferred secondary drying methods include vacuum drying, or tray drying under ambient conditions. To minimize chemical degradation during drying, drying may take place under an inert gas such as nitrogen, or may take place under vacuum.

The solid amorphous dispersion may be in the form of small particles. The mean size of the particles may be less than 500 μm in diameter, or less than 100 μm in diameter, less than 50 μm in diameter or less than 25 μm in diameter. When the solid amorphous dispersion is formed by spray-drying, the resulting dispersion is in the form of such small particles. When the solid amorphous dispersion is formed by other methods such by melt-congeal or extrusion processes, the resulting dispersion may be sieved, ground, milled, or otherwise processed to yield a plurality of small particles.

For ease of processing, the dried particles may have certain density and size characteristics. In one embodiment, the resulting solid amorphous dispersion particles are formed by spray drying and may have a bulk specific volume of less than or equal to about 4 cc/g, and more preferably less than or equal to about 3.5 cc/g. The particles may have a tapped specific volume of less than or equal to about 3 cc/g, and more preferably less than or equal to about 2 cc/g. The particles have a Hausner ratio of less than or equal to about 3, and more preferably less than or equal to about 2. The particles may have a mean particle diameter up to about 150 μm, and more preferably from about 1 to about 25 μm. The particles may have a Span of less than or equal to 3, and more preferably less than or equal to about 2.5. As used herein, “Span,” is defined as

$$Span = \frac{{D}_{90} - {D}_{10}}{{D}_{50}} ,$$

where D₁₀ is the diameter corresponding to the diameter of particles that make up 10% of the total volume containing particles of equal of smaller diameter, D₅₀ is the diameter corresponding to the diameter of particles that make up 50% of the total volume containing particles of equal or smaller diameter, and D₉₀ is the diameter corresponding to the diameter of particles that make up 90% of the total volume containing particles of equal or smaller diameter.

Any of the processes previously listed as appropriate for forming dispersions may be used to form amorphous Compound A. In particular, amorphous Compound A may be made by dissolving Compound A in a solvent such as acetone or methanol and spray-drying in generally the same manner in which is described above for making dispersions of Compound A in matrix. Amorphous Compound A may also be made, for example, by feeding crystalline Compound A to a melt congeal apparatus such as that disclosed in U.S. Pat. No. 5,183,493 or 5,549,917 such that droplets of molten Compound A are formed and then cooled by a cooling gas to form amorphous particles of Compound A ranging from about 1 to about 500 μm in diameter and preferably about 10 to 300 μm in diameter.

The solid amorphous dispersion or amorphous drug may be stored in an enclosed package to reduce exposure to humidity (water vapor) and/or oxygen. Exemplary methods for reducing contact with humidity or oxygen include protective packaging that is substantially impermeable to water vapor and/or oxygen, such as foil pouches or foil blister packs, or inclusion of a desiccant or an oxygen absorber such as oxygen adsorbent packets in the packaging for the compositions. The packaging may include a desiccant to reduce humidity, an oxygen absorber, or both. Exemplary desiccants include aluminosilicate (Sorb-it®, available from Süd-Chemie), and anhydrous calcium sulfate, and exemplary oxygen absorbers include oxygen absorbing compositions disclosed in U.S. Pat. No. 6,558,571 and packets and strips sold under the trade name Fresh Pax™ available from Multisorb Technologies, Inc. The present invention provides pharmaceutical packages comprising a pharmaceutical composition comprising (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, said package further comprising an oxygen absorber. In a further aspect are provided such packages, wherein the amount of oxygen gas present is less than about 5% by volume of the total amount of gas present in said package. In further aspects, the amount of oxygen gas present is less than about 2%, or less than about 1%, or less than about 0.5%, or less than about 0.25%, or less than about 0.1%, or less than about 0.01% of the total amount of gas present in said package.

### Concentration Enhancement

In a preferred embodiment, the compositions of the present invention provide concentration enhancement when dosed to an aqueous environment of use, meaning that they meet at least one of the following conditions. The first condition is that the inventive compositions increase the maximum dissolved concentration (MDC) of Compound A in the environment of use relative to a control composition consisting of an equivalent amount of crystalline Form I of Compound A. That is, once the composition is introduced into an environment of use, the composition increases the aqueous concentration of Compound A relative to the control composition. It is to be understood that the control composition is free from solubilizers or other components that would materially affect the solubility of crystalline Form I of Compound A, and that Compound A is in solid Form In the control composition. The control composition is conventionally the undispersed crystalline Form I of Compound A alone. Preferably, the inventive compositions provide an MDC of Compound A in aqueous solution that is at least 1.25-fold, or at least 2-fold, or at least 3-fold, that provided by the control composition. In some cases, the MDC of Compound A provided by the compositions of the present invention is at least 5-fold or at least 10-fold the equilibrium concentration provided by the control composition.

The second condition is that the inventive compositions increase the dissolution area under the concentration versus time curve (AUC) of Compound A in the environment of use relative to a control composition consisting of an equivalent amount of crystalline Form I of Compound A with no polymer. The calculation of an AUC is a well-known procedure in the pharmaceutical arts and is described, for example, in Welling, “Pharmacokinetics Processes and Mathematics,” ACS Monograph 185 (1986). More specifically, in the environment of use, the inventive compositions provide an AUC for any 90-minute period of from about 0 to about 270 minutes following introduction to the use environment that is at least about 1.25-fold that of the control composition described above. Preferably, the AUC provided by the composition is at least about 1.25-fold, or at least about 2-fold, or at least about 3-fold, that of the control composition. Some compositions of the present invention may provide an AUC value that is at least about 5-fold or at least about 10-fold that of a control composition as described above.

In one embodiment of the present invention, the composition comprises amorphous Compound A and a concentration-enhancing polymer in an amount sufficient such that the composition provides concentration enhancement relative to a second control composition consisting of amorphous Compound A without a concentration-enhancing polymer. In these compositions, the polymer enhances at least one of the MDC or AUC of Compound A in aqueous solution by at least 1.25-fold, or at least about 2-fold, or at least about 3-fold, relative to the second control composition.

As previously mentioned, a “use environment” can be either the in vivo environment, such as the GI tract of an animal, particularly a human, or the in vitro environment of a test solution, such as phosphate buffered saline (PBS) solution or Model Fasted Duodenal (MFD) solution. The inventors have found that in vitro dissolution tests are good predictors of in vivo behavior, and thus compositions are within the scope of the invention if they provide concentration-enhancement in either or both in vitro and in vivo use environments.

The compositions of the present invention provide enhanced concentration of the dissolved Compound A in in vitro dissolution tests. It has been determined that enhanced drug concentration in in vitro dissolution tests in MFD solution or in PBS solution is a good indicator of in vivo performance and bioavailability. An appropriate PBS solution is an aqueous solution comprising 20 mM Na₂HPO₄, 47 mM KH₂PO₄, 87 mM NaCl, and 0.2 mM KCl, adjusted to pH 6.5 with NaOH. An appropriate MFD solution is the same PBS solution wherein there is also present 7.3 mM sodium taurocholic acid and 1.4 mM of 1-palmitoyl-2-oleyl-sn-glycero-3-phosphocholine. In particular, a composition formed by the inventive method can be dissolution-tested by adding it to MFD or PBS solution and agitating to promote dissolution.

An in vitro test to evaluate enhanced Compound A concentration in aqueous solution can be conducted by (1) adding with agitation a sufficient quantity of control composition, typically the undispersed Compound A alone, to the in vitro test medium, such as an MFD or a PBS solution, to achieve equilibrium concentration of Compound A; (2) in a separate test, adding with agitation a sufficient quantity of test composition (e.g., the composition comprising Compound A and polymer) in the same test medium, such that if all Compound A dissolved, the theoretical concentration of Compound A would exceed the equilibrium concentration of Compound A by a factor of at least 2 or a factor of at least 10; and (3) comparing the measured MDC and/or aqueous AUC of the test composition in the test medium with the equilibrium concentration, and/or with the aqueous AUC of the control composition. In conducting such a dissolution test, the amount of test composition or control composition used is an amount such that if all of Compound A dissolved the Compound A concentration would be at least 2-fold, preferably at least 10-fold, and most preferably at least 100-fold that of the equilibrium concentration.

The concentration of dissolved Compound A is typically measured as a function of time by sampling the test medium and plotting Compound A concentration in the test medium vs. time so that the MDC can be ascertained. The MDC is taken to be the maximum value of dissolved Compound A measured over the duration of the test. The aqueous AUC is calculated by integrating the concentration versus time curve over any 90-minute time period between the time of introduction of the composition into the aqueous use environment (when time equals zero) and 270 minutes following introduction to the use environment (when time equals 270 minutes). Typically, when the composition reaches its MDC rapidly, in say less than about 30 minutes, the time interval used to calculate AUC is from time equals zero to time equals 90 minutes. However, if the AUC of a composition over any 90-minute time period described above meets the criterion of this invention, then the composition formed is considered to be within the scope of this invention.

To avoid large drug particulates that would give an erroneous determination, the test solution is either filtered or centrifuged. “Dissolved drug” is typically taken as that material that either passes a 0.45 μm syringe filter or, alternatively, the material that remains in the supernatant following centrifugation. Filtration can be conducted using a 13 mm, 0.45 μm polyvinylidine difluoride syringe filter sold by Scientific Resources under the trademark TITAN®. Centrifugation is typically carried out in a polypropylene microcentrifuge tube by centrifuging at 13,000 G for 60 seconds. Other similar filtration or centrifugation methods can be employed and useful results obtained. For example, using other types of microfilters may yield values somewhat higher or lower (±10-40%) than that obtained with the filter specified above but will still allow identification of preferred dispersions. It should be recognized that this definition of “dissolved drug” encompasses not only monomeric solvated drug molecules but also a wide range of species such as polymer/Compound A assemblies that have submicron dimensions such as Compound A aggregates, aggregates of mixtures of polymer and drug, micelles, polymeric micelles, colloidal particles or nanocrystals, polymer/drug complexes, and other such drug-containing species that are present in the filtrate or supernatant in the specified dissolution test.

Furthermore, the compositions of the present invention, when dosed orally to a human or other mammal in the fasted state, provide a Cₘₐₓ of at least about 1.25-fold, or at least about 2-fold, or at least about 3-fold, or at least about 5-fold, or at least about 10-fold that provided by a control composition consisting of an equivalent quantity of crystalline Form I of Compound alone.

Alternatively, the compositions, when dosed orally to a human or other mammal in the fasted state, provide an AUC in Compound A concentration in the blood (serum or plasma) that is at least about 1.25-fold, or at least about 2-fold, or at least about 3-fold, or at least about 5-fold, or at least about 10-fold that observed when a control composition consisting of an equivalent quantity of crystalline Form I of Compound A is dosed alone without any additional polymer. It is noted that such compositions can also be said to have a relative bioavailability of from about 1.25-fold to about 10-fold that of the control composition.

Relative bioavailability of Compound A in the compositions can be tested in vivo in animals or humans using conventional methods for making such a determination. An in vivo test, such as a crossover study, may be used to determine whether a composition of Compound A and matrix provides an enhanced relative bioavailability compared with a control composition as described above. In an in vivo crossover study a test composition of a solid amorphous dispersion of Compound A and matrix is dosed to half a group of test subjects and, after an appropriate washout period (e.g., one week) the same subjects are dosed with a control composition that consists of an equivalent quantity of crystalline Form I of Compound A as the test composition (but with no matrix present). The other half of the group is dosed with the control composition first, followed by the test composition. The relative bioavailability is measured as the concentration in the blood (serum or plasma) versus time area under the curve (AUC) determined for the test group divided by the AUC in the blood provided by the control composition. Preferably, this test/control ratio is determined for each subject, and then the ratios are averaged over all subjects in the study. In vivo determinations of AUC can be made by plotting the serum or plasma concentration of Compound A along the ordinate (y-axis) against time along the abscissa (x-axis). To facilitate dosing, a dosing vehicle may be used to administer the dose. The dosing vehicle is preferably water, but may also contain materials for suspending the test or control composition, provided these materials do not dissolve the composition or change Compound A solubility in vivo.

### Dosage Forms

The compositions may be delivered by a wide variety of routes, including, but not limited to, oral, nasal, rectal, vaginal, subcutaneous, intravenous and pulmonary. Generally, the oral route is preferred.

The compositions may also be used in a wide variety of dosage forms for administration of drugs. Exemplary dosage forms are powders or granules that may be taken orally either dry or reconstituted by addition of water or other liquids to form a paste, slurry, suspension or solution; tablets; capsules; multiparticulates; and pills. Various additives may be mixed, ground, or granulated with the compositions of this invention to form a material suitable for the above dosage forms.

The compositions of the present invention may be formulated in various forms such that they are delivered as a suspension of particles in a liquid vehicle. Such suspensions may be formulated as a liquid or paste at the time of manufacture, or they may be formulated as a dry powder with a liquid, typically water, added at a later time but prior to oral administration. Such powders that are constituted into a suspension are often termed sachets or oral powder for constitution (OPC) formulations. Such dosage forms can be formulated and reconstituted via any known procedure. The simplest approach is to formulate the dosage form as a dry powder that is reconstituted by simply adding water and agitating. Alternatively, the dosage form may be formulated as a liquid and a dry powder that are combined and agitated to form the oral suspension. In yet another embodiment, the dosage form can be formulated as two powders that are reconstituted by first adding water to one powder to form a solution to which the second powder is combined with agitation to form the suspension.

Additionally, the compositions of the present invention may be administered in combination with an additional agent or agents for the treatment of a mammal, such as a human, that is suffering from an infection with the HIV virus, AIDS, AIDS-related complex (ARC), or any other disease or condition which is related to infection with the HIV virus. The agents that may be used in combination with the compositions of the present invention include, but are not limited to, those useful as HIV protease inhibitors, HIV reverse transcriptase inhibitors, non-nucleoside HIV reverse transcriptase inhibitors, inhibitors of HIV integrase, CCR5 inhibitors, HIV fusion inhibitors, compounds useful as immunomodulators, compounds that inhibit the HIV virus by an unknown mechanism, compounds useful for the treatment of herpes viruses, compounds useful as anti-infectives, and others as described below.

Compounds useful as HIV protease inhibitors that may be used in combination with the compositions of the present invention include, but are not limited to, 141 W94 (amprenavir), CGP-73547, CGP-61755, DMP-450, nelfinavir, ritonavir, saquinavir (invirase), lopinavir, TMC-126, atazanavir, palinavir, GS-3333, KN I-413, KNI-272, LG-71350, CGP-61755, PD 173606, PD 177298, PD 178390, PD 178392, U-140690, ABT-378, DMP-450, AG-1776, MK-944, VX-478, indinavir, tipranavir, TMC-114, DPC-681, DPC-684, fosamprenavir calcium (Lexiva), benzenesulfonamide derivatives disclosed in WO 03053435, R-944, Ro-03-34649, VX-385, GS-224338, OPT-TL3, PL-100, SM-309515, AG-148, DG-35-VIII, DMP-850, GW-5950×, KNI-1039, L-756423, LB-71262, LP-130, RS-344, SE-063, UIC-94-003, Vb-19038, A-77003, BMS-182193, BMS-186318, SM-309515, JE-2147, GS-9005.

Compounds useful as inhibitors of the HIV reverse transcriptase enzyme that may be used in combination with the compositions of the present invention include, but are not limited to, abacavir, FTC, GS-840, lamivudine, adefovir dipivoxil, beta-fluoro-ddA, zalcitabine, didanosine, stavudine, zidovudine, tenofovir, amdoxovir, SPD-754, SPD-756, racivir, reverset (DPC-817), MIV-210 (FLG), beta-L-Fd4C (ACH-126443), MIV-310 (alovudine, FLT), dOTC, DAPD, entecavir, GS-7340, emtricitabine, alovudine.

Compounds useful as non-nucleoside inhibitors of the HIV reverse transcriptase enzyme include, but are not limited to, efavirenz, HBY-097, nevirapine, TMC-120 (dapivirine), TMC-125, etravirine, delavirdine, DPC-083, DPC-961, TMC-120, capravirine, GW-678248, GW-695634, calanolide, and tricyclic pyrimidinone derivatives as disclosed in WO 03062238.

Compounds useful as CCR5 inhibitors that may be used in combination with the compositions of the present invention include, but are not limited to, TAK-779, SC-351125, SCH-D, UK-427857, PRO-140, and GW-873140 (Ono-4128, AK-602).

Compounds useful as inhibitors of HIV integrase enzyme that may be used in combination with the compositions of the present invention include, but are not limited to, GW-810781, 1,5-naphthyridine-3-carboxamide derivatives disclosed in WO 03062204, compounds disclosed in WO 03047564, compounds disclosed in WO 03049690, and 5-hydroxypyrimidine-4-carboxamide derivatives disclosed in WO 03035076.

Fusion inhibitors for the treatment of HIV that may be used in combination with the compositions of the present invention include, but are not limited to, enfuvirtide (T-20), T-1249, AMD-3100, and fused tricyclic compounds disclosed in JP 2003171381.

Other compounds that are useful inhibitors of HIV that may be used in combination with the compositions of the present invention include, but are not limited to, Soluble CD4, TNX-355, PRO-542, BMS-806, tenofovir disoproxil fumarate, and compounds disclosed in JP 2003119137.

Compounds useful in the treatment or management of infection from viruses other than HIV that may be used in combination with the compositions of the present invention include, but are not limited to, acyclovir, fomivirsen, penciclovir, HPMPC, oxetanocin G, AL-721, cidofovir, cytomegalovirus immune globin, cytovene, fomivganciclovir, famciclovir, foscarnet sodium, Isis 2922, KNI-272, valacyclovir, virazole ribavirin, valganciclovir, ME-609, PCL-016.

Compounds that act as immunomodulators and may be used in combination with the compositions of the present invention include, but are not limited to, AD-439, AD-519, Alpha Interferon, AS-101, bropirimine, acemannan, CL246,738, EL10, FP-21399, gamma interferon, granulocyte macrophage colony stimulating factor, IL-2, immune globulin intravenous, IMREG-1, IMREG-2, imuthiol diethyl dithio carbamate, alpha-2 interferon, methionine-enkephalin, MTP-PE, granulocyte colony stimulating sactor, remune, rCD4, recombinant soluble human CD4, interferon alfa-2, SK&F106528, soluble T4 yhymopentin, tumor necrosis factor (TNF), tucaresol, recombinant human interferon beta, and interferon alfa n-3.

Anti-infectives that may be used in combination with the compositions of the present invention include, but are not limited to, atovaquone, azithromycin, clarithromycin, trimethoprim, trovafloxacin, pyrimethamine, daunorubicin, clindamycin with primaquine, fluconazole, pastill, ornidyl, eflornithine pentamidine, rifabutin, spiramycin, intraconazole-R51211, trimetrexate, daunorubicin, recombinant human erythropoietin, recombinant human growth hormone, megestrol acetate, testerone, and total enteral nutrition.

Antifungals that may be used in combination with the compositions of the present invention include, but are not limited to, anidulafungin, C31G, caspofungin, DB-289, fluconzaole, itraconazole, ketoconazole, micafungin, posaconazole, and voriconazole.

Other compounds that may be used in combination with the compositions of the present invention include, but are not limited to, acmannan, ansamycin, LM 427, AR177, BMS-232623, BMS-234475, CI-1012, curdlan sulfate, dextran sulfate, STOCRINE EL10, hypericin, lobucavir, novapren, peptide T octabpeptide sequence, trisodium phosphonoformate, probucol, and RBC-CD4.

In addition, the compositions of the present invention may be used in combination with anti-proliferative agents for the treatment of conditions such as Kaposi's sarcoma. Such agents include, but are not limited to, inhibitors of metallo-matrix proteases, A-007, bevacizumab, BMS-275291, halofuginone, interleukin-12, rituximab, paclitaxel, porfimer sodium, rebimastat, and COL-3.

The particular choice of an additional agent or agents will depend on a number of factors that include, but are not limited to, the condition of the mammal being treated, the particular condition or conditions being treated, the identity of the additional agent or agents, and the identity of any additional compounds that are being used to treat the mammal. The particular choice of the additional agent or agents is within the knowledge of one of ordinary skill in the art.

The compositions of the present invention may be administered in combination with any of the above additional agents for the treatment of a mammal, such as a human, that is suffering from an infection with the HIV virus, AIDS, AIDS-related complex (ARC), or any other disease or condition which is related to infection with the HIV virus. Such a combination may be administered to a mammal such that the compositions of the present invention are present in the same formulation as the additional agents described above. Alternatively, such a combination may be administered to a mammal suffering from infection with the HIV virus such that the compositions of the present invention are present in a formulation that is separate from the formulation in which the additional agent is found. If the compositions of the present invention are administered separately from the additional agent, such administration may take place concomitantly or sequentially with an appropriate period of time in between. The choice of whether to include the compositions of the present invention in the same formulation as the additional agent or agents is within the knowledge of one of ordinary skill in the art.

Additionally, the compositions of the present invention may be administered to a mammal, such as a human, in combination with an additional agent that has the effect of increasing the exposure of the mammal to a compound of the invention. The term “exposure,” as used herein, refers to the concentration of a compound of the invention in the plasma of a mammal as measured over a period of time. The exposure of a mammal to Compound A can be measured by administering a composition of the invention to a mammal in an appropriate form, withdrawing plasma samples at predetermined times, and measuring the amount of Compound A in the plasma using an appropriate analytical technique, such as liquid chromatography or liquid chromatography/mass spectroscopy. The amount of Compound A in the plasma at a certain time is determined and the concentration and time data from all the samples are plotted to afford a curve. The area under this curve is calculated and affords the exposure of the mammal to the compound. The terms “exposure,” “area under the curve,” and “area under the concentration/time curve” are intended to have the same meaning and may be used interchangeably throughout.

Among the agents that may be used to increase the exposure of a mammal to Compound A are those that can act as inhibitors of at least one isoform of the cytochrome P450 (CYP450) enzymes. The isoforms of CYP450 that may be beneficially inhibited include, but are not limited to, CYP1A2, CYP2D6, CYP2C9, CYP2C19 and CYP3A4. Suitable agents that may be used to inhibit CYP 3A4 include, but are not limited to, ritonavir and delavirdine.

Such a combination may be administered to a mammal such that Compound A is present in the same formulation as the additional agents described above. Alternatively, such a combination may be administered such that Compound A is present in a formulation that is separate from the formulation in which the additional agent is found. If Compound A is administered separately from the additional agent, such administration may take place concomitantly or sequentially with an appropriate period of time in between. The choice of whether to include compositions of the present invention in the same formulation as the additional agent or agents is within the knowledge of one of ordinary skill in the art.

Other features and embodiments of the invention will become apparent from the following examples that are given for illustration of the invention rather than for limiting its intended scope.

### EXAMPLES

### Example 1

Example 1, consisting of amorphous Compound A, was prepared as follows. First, a spray solution was formed containing 250.1 mg Compound A and 24.5 g methanol. The solution was pumped into a “mini” spray-drying apparatus via a Cole Parmer 74900 series rate-controlling syringe pump at a rate of 70 mL/hr. Compound A solution was atomized through a Spraying Systems Co. two-fluid nozzle, Model No. SU1A using a heated stream of nitrogen at a flow rate of 1 SCFM. The spray solution was sprayed into an 11-cm diameter stainless steel chamber. The heated gas entered the chamber at an inlet temperature of 100° C. and exited at an outlet temperature of 22° C. The resulting amorphous Compound A was collected on filter paper, dried under vacuum, and stored in a desiccator. The yield was about 78%.

An in vitro dissolution test was performed to determine whether the amorphous Compound A of Example 1 provides concentration-enhancement relative to the crystalline form of Compound A. For this test, a sufficient amount of material was added to a microcentrifuge test tube so that the concentration of Compound A would have been 3000 μg/mL, if all of Compound A had dissolved. The test was run in duplicate. The tubes were placed in a 37° C. temperature-controlled chamber, and 1.8 mL PBS at pH 6.5 and 290 mOsm/kg was added to each respective tube. The samples were quickly mixed using a vortex mixer for about 60 seconds. The samples were centrifuged at 13,000 G at 37° C. for 1 minute. The resulting supernatant solution was then sampled and diluted 1:6 (by volume) with methanol and then analyzed by high-performance liquid chromatography (HPLC). HPLC analysis was performed using a Phenomenex Luna C₁₈ column. The mobile phase consisted of 55% 20 mM KH₂PO₄, adjusted to pH 3 with H₃PO₄, and 45% acetonitrile. UV absorbance was measured at 210 nm. The contents of each tube were mixed on the vortex mixer and allowed to stand undisturbed at 37° C. until the next sample was taken. Samples were collected at 4, 10, 20, 40, 90 and 1200 minutes. The results are shown in Table 1.

Control 1

Control 1 consisted of crystalline Form I of Compound A alone, and a sufficient amount of material was added so that the concentration of Compound A would have been 3000 μg/mL, if all of Compound A had dissolved.

TABLE 1

| | | Compound A | |
|---|---|---|---|
| | Time | Concentration | AUC |
| | (min) | (μg/mL) | (min * μg/mL) |
| Example 1 | 0 | 0 | 0 |
| (amorphous | 4 | 1070 | 2100 |
| Compound A) | 10 | 1140 | 8800 |
| | 20 | 1150 | 20,200 |
| | 40 | 1150 | 43,200 |
| | 90 | 158 | 75,900 |
| | 1200 | 126 | 233,200 |
| Control 1 | 0 | 0 | 0 |
| (crystalline Form | 4 | 100 | 200 |
| I of Compound | 10 | 100 | 800 |
| A) | 20 | 106 | 1800 |
| | 40 | 101 | 3900 |
| | 90 | 112 | 9200 |
| | 1200 | 109 | 131,700 |

The concentrations of drug obtained in these samples were used to determine the maximum concentration of drug (“MDC₉₀”), the area under the concentration-versus-time curve (“AUC₉₀”) during the initial ninety minutes, and the Compound A concentration at 1200 minutes (“C₁₂₀₀”). The results are shown in Table 2.

TABLE 2

| | MDC₉₀ | AUC₉₀ | C₁₂₀₀ |
|---|---|---|---|
| | (μg/mL) | (min * μg/mL) | (μg/mL) |
| Example 1 | 1153 | 75,900 | 126 |
| (amorphous | | | |
| Compound A) | | | |
| Control 1 | 112 | 9200 | 109 |
| (crystalline Form | | | |
| I of | | | |
| Compound A) | | | |

The results show that the amorphous form of Compound A provides concentration-enhancement relative to crystalline Form I of Compound A alone. The amorphous form of Compound A provided a MDC that was 10.3-fold and an AUC₉₀ that was 8.3-fold that provided by crystalline Form I of Compound A.

### Example 2

Example 2 was prepared by combining amorphous Compound A and a concentration-enhancing polymer. A simple physical mixture of Compound A and the concentration-enhancing polymer hydroxypropyl methyl cellulose acetate succinate (AQUOT-MG, available from Shin Etsu, Tokyo, Japan) was prepared by adding 5.4 mg of amorphous Compound A prepared as in Example 1 and 0.6 mg of HPMCAS to a centrifuge tube. The dry powders were mixed using a vortex mixer for 1 minute. Dissolution tests were performed as described in Example 1. The results are shown in Table 3.

TABLE 3

| | | Compound A | |
|---|---|---|---|
| | Time | Concentration | AUC |
| Example | (min) | (μg/mL) | (min * μg/mL) |
| Example 2 | 0 | 0 | 0 |
| | 4 | 681 | 1400 |
| | 10 | 764 | 5700 |
| | 20 | 775 | 13,400 |
| | 40 | 888 | 30,000 |
| | 90 | 956 | 76,100 |
| | 1200 | 979 | 1,150,000 |

The concentrations of drug obtained in these samples were used to determine the maximum concentration of drug, MDC₉₀, the area under the concentration-versus-time curve during the initial ninety minutes, AUC₉₀, and the Compound A concentration at 1200 minutes, C₁₂₀₀. The results are shown in Table 4. Example 1 and Control 1 are shown again for comparison.

TABLE 4

| | MDC₉₀ | AUC₉₀ | C₁₂₀₀ |
|---|---|---|---|
| Example | (μg/mL) | (min * μg/mL) | (μg/mL) |
| Ex 2 | 956 | 76,100 | 979 |
| Ex 1 | 1150 | 75,900 | 126 |
| Control 1 | 112 | 9200 | 109 |

The physical mixture of Example 2 containing both amorphous Compound A and the concentration-enhancing polymer had improved dissolution performance relative to crystalline Form I Compound A alone (Control 1). Example 2 provided a MDC₉₀ that was 8.7-fold that provided by crystalline Form I Compound A alone, and an AUC₉₀ that was 8.3-fold that provided by crystalline Form I Compound A alone.

Example 2 also sustained dissolved drug concentration for a longer time than amorphous Compound A alone. While the performance of amorphous Compound A alone (Example 1) and amorphous Compound A plus concentration-enhancing polymer (Example 2) had similar performance during the initial ninety minutes, addition of the concentration-enhancing polymer substantially improved dissolution performance of Compound A at later times. Example 2 provided a dissolved Compound A concentration at 1200 minutes (“C₁₂₀₀”) that was 7.8-fold that of amorphous Compound A alone.

### Example 3

Example 3, a solid amorphous dispersion containing 90 wt % Compound A and 10 wt % HPMCAS (AQUOT-MG, available from Shin Etsu, Tokyo, Japan), was prepared as follows. First, a spray solution was formed containing 300 g Compound A, 33.3 g HPMCAS, and 3000 g methanol as follows. The HPMCAS and methanol were combined in a container and mixed for about 2 hours, allowing the HPMCAS to dissolve. The resulting mixture had a slight haze after the entire amount of polymer had been added. Next, Compound A was added directly to this mixture, and the mixture stirred for an additional 2 hours. This mixture was then filtered by passing it through a filter with a screen size of 250 μm to remove any large insoluble material from the mixture, thus forming the spray solution.

The spray solution was pumped using a high-pressure pump to a spray drier (a Niro type XP Portable Spray-Dryer with a Liquid-Feed Process Vessel (“PSD-1”)), equipped with a pressure nozzle (Spraying Systems Pressure Nozzle and Body) (SK 76-16). The PSD-1 was equipped with a 9-inch chamber extension. The 9-inch chamber extension was added to the spray dryer to increase the vertical length of the dryer. The added length increased the residence time within the dryer, which allowed the product to dry before reaching the angled section of the spray dryer. The spray drier was also equipped with a 316 SS circular diffuser plate with 1/16-inch drilled holes, having a 1% open area. This small open area directed the flow of the drying gas to minimize product recirculation within the spray dryer. The nozzle sat flush with the diffuser plate during operation. A Bran+Lubbe high-pressure pump was used to deliver liquid to the nozzle. The pump was followed by a pulsation dampener to minimize pulsation at the nozzle. The spray solution was pumped to the spray drier at about 180 g/min at a pressure of 200 psig. Drying gas (e.g., nitrogen) was circulated through the diffuser plate at an inlet temperature of 200° C. The evaporated solvent and drying gas exited the spray drier at a temperature of 60° C. The resulting solid amorphous dispersion was collected in a cyclone.

The solid amorphous dispersion formed using the above procedure was post-dried using a Gruenberg single-pass convection tray dryer operating at 40° C. for 6 hours. Following drying, the dispersion was then equilibrated with ambient air and humidity (20° C./50% RH) for 8 hours.

The properties of the solid amorphous dispersion after secondary drying are shown in Table 5.

TABLE 5

| Bulk Properties (After Secondary Drying) | Tray Dried @ 40° C. |
|---|---|
| Bulk Specific Volume (cc/g) | 3.0 |
| Tapped Specific Volume (cc/g) | 1.7 |
| Hausner Ratio | 1.72 |
| Mean Particle Diameter (μm) | 17 |
| D₁₀, D₅₀, D₉₀* (μm) | 4, 14, 33 |
| Span (D₉₀ − D₁₀)/D₅₀ | 1.97 |
| Residual Methanol | 2.0% |
| (Before Secondary Drying) | |

*10 vol % of the particles have a diameter that is smaller than D₁₀; 50 vol % of the particles have a diameter that is smaller than D₅₀, and 90 vol % of the particles have a diameter that is smaller than D₉₀.

### Examples 4-11

Solid amorphous dispersions of Compound A were made with various ratios of Compound A to concentration-enhancing polymer and various concentration-enhancing polymers, using a “mini” spray-drying apparatus. Table 6 lists the concentration of Compound A in each dispersion and the concentration-enhancing polymers used.

TABLE 6

| | Compound A Concentration | |
|---|---|---|
| Example | in Dispersion | |
| No. | (active, wt %) | Polymer* |
| 4 | 40 | HPMCAS-MF |
| 5 | 50 | HPMCAS-MF |
| 6 | 75 | HPMCAS-MF |
| 7 | 90 | HPMCAS-MF |
| 8 | 95 | HPMCAS-MG |
| 9 | 98 | HPMCAS-MG |
| 10 | 50 | CMEC |
| 11 | 50 | HPMC |

*Polymer designations:
HPMCAS-MF = hydroxypropyl methyl cellulose acetate succinate (AQUOT MF grade, available from Shin Etsu, Tokyo, Japan CHECK),
CMEC = carboxymethyl ethylcellulose (available from Freund, Tokyo, Japan),
HPMC = hydroxypropyl methyl cellulose (E3 Prem Grade available from Dow Chemical Co., Midland, MI)

To prepare dispersions using the mini spray drier, Compound A was mixed in a solvent together with the polymer to form a spray solution. Each solution was pumped into a “mini” spray-drying apparatus via a Cole Parmer 74900 series rate-controlling syringe pump. The Compound A/polymer solution was atomized through a Spraying Systems Co. two-fluid nozzle, model No. SU1A using a heated stream of nitrogen (100° C.). The spray solution was sprayed into an 11-cm diameter stainless steel chamber. The resulting solid amorphous dispersion was collected on filter paper, dried under vacuum, and stored in a desiccator. The conditions used to spray-dry each dispersion are listed in Table 7.

TABLE 7

| | Compound | | Polymer | | Solvent | Feed | |
|---|---|---|---|---|---|---|---|
| Example | A Mass | | Mass | | Mass | Rate | |
| No. | (mg) | Polymer | (mg) | Solvent | (g) | (mL/min) | Tᵢₙ (° C.) |
| 4 | 160 | HPMCAS-MF | 240 | acetone | 20 | 1.3 | 100 |
| 5 | 250.6 | HPMCAS-MF | 250.6 | acetone | 24.5 | 1.3 | 100 |
| 6 | 300 | HPMCAS-MF | 100 | acetone | 20 | 1.3 | 100 |
| 7 | 250 | HPMCAS-MF | 27.8 | acetone | 25.3 | 1.2 | 100 |
| 8 | 475 | HPMCAS-MG | 25 | MeOH | 24.5 | 1.3 | 70 |
| 9 | 490 | HPMCAS-MG | 10 | MeOH | 24.5 | 1.3 | 70 |
| 10 | 250 | CMEC | 250.3 | acetone | 24.5 | 1.3 | 100 |
| 11 | 250.7 | HPMC | 250.6 | MeOH/H₂O 9/1* | 25 | 1.0 | 110 |

*(volume/volume)

### Example 12

Dissolution tests were performed to demonstrate that the solid amorphous dispersions of Examples 3-11 provide concentration-enhancement of Compound A. In vitro dissolution tests were performed as in Example 1. For these tests, a sufficient amount of material was added so that the concentration of Compound A would have been 3000 μg/mL, if all of Compound A had dissolved. The results are shown in Table 8.

TABLE 8

| | | Compound A | |
|---|---|---|---|
| | Time | Concentration | AUC |
| Example | (min) | (μg/mL) | (min * μg/mL) |
| 3 | 0 | 0 | 0 |
| | 4 | 970 | 1900 |
| | 10 | 1040 | 8000 |
| | 20 | 1080 | 18,600 |
| | 40 | 1140 | 40,800 |
| | 90 | 1060 | 95,900 |
| | 1200 | 1080 | 1,280,000 |
| 4 | 0 | 0 | 0 |
| | 4 | 1330 | 2700 |
| | 10 | 1400 | 10,900 |
| | 20 | 1520 | 25,500 |
| | 40 | 1550 | 56,100 |
| | 90 | 1580 | 134,400 |
| | 1200 | 1610 | 1,900,000 |
| 5 | 0 | 0 | 0 |
| | 4 | 1190 | 2400 |
| | 10 | 1280 | 9800 |
| | 20 | 1360 | 22,900 |
| | 40 | 1370 | 50,200 |
| | 90 | 1450 | 120,500 |
| | 1200 | 1490 | 1,750,000 |
| 6 | 0 | 0 | 0 |
| | 4 | 1370 | 2700 |
| | 10 | 1200 | 10,400 |
| | 20 | 1220 | 22,500 |
| | 40 | 1220 | 46,900 |
| | 90 | 1350 | 111,000 |
| | 1200 | 1440 | 1,660,000 |
| 7 | 0 | 0 | 0 |
| | 4 | 989 | 2000 |
| | 10 | 1060 | 8100 |
| | 20 | 1030 | 18,600 |
| | 40 | 1110 | 40,000 |
| | 90 | 1180 | 97,200 |
| | 1200 | 1240 | 1,430,000 |
| 8 | 0 | 0 | 0 |
| | 4 | 997 | 2000 |
| | 10 | 1080 | 8200 |
| | 20 | 1120 | 19,200 |
| | 40 | 1170 | 42,100 |
| | 90 | 1210 | 101,400 |
| | 1200 | 1160 | 1,420,000 |
| 9 | 0 | 0 | 0 |
| | 4 | 935 | 1900 |
| | 10 | 1030 | 7800 |
| | 20 | 1110 | 18,500 |
| | 40 | 1140 | 40,900 |
| | 90 | 1130 | 97,500 |
| | 1200 | 1200 | 1,390,000 |
| 10 | 0 | 0 | 0 |
| | 4 | 1430 | 2900 |
| | 10 | 1440 | 11,400 |
| | 20 | 1410 | 25,700 |
| | 40 | 1410 | 53,800 |
| | 90 | 1440 | 125,100 |
| | 1200 | 1450 | 1,730,000 |
| 11 | 0 | 0 | 0 |
| | 4 | 1130 | 2300 |
| | 10 | 1150 | 9100 |
| | 20 | 1150 | 20,600 |
| | 40 | 1200 | 44,100 |
| | 90 | 1150 | 102,900 |
| | 1200 | 1190 | 1,400,000 |

The concentrations of Compound A obtained in these samples were used to determine the maximum concentration of drug (“MDC₉₀”) and the area under the concentration-versus-time curve (“AUC₉₀”) during the initial ninety minutes. The results are shown in Table 5. The results for Example 1 (amorphous Compound A) and Control 1 (crystalline Form I Compound A) are shown for comparison.

TABLE 9

| | Compound A | | | | | |
|---|---|---|---|---|---|---|
| | Concentration in | | | | | |
| | Dispersion | | Dose | MDC₉₀ | AUC₉₀ | AUC₁₂₀₀ |
| Example | (active, wt %) | Polymer | (μg/mL) | (μg/mL) | (min*μg/mL) | (min*μg/mL) |
| 3 | 90 | HPMCAS | 3000 | 1140 | 95,900 | 1,280,000 |
| 4 | 40 | HPMCAS | 3000 | 1580 | 134,400 | 1,900,000 |
| 5 | 50 | HPMCAS | 3000 | 1450 | 120,500 | 1,750,000 |
| 6 | 75 | HPMCAS | 3000 | 1370 | 111,000 | 1,660,000 |
| 7 | 90 | HPMCAS | 3000 | 1180 | 97,200 | 1,430,000 |
| 8 | 95 | HPMCAS | 3000 | 1210 | 101,400 | 1,420,000 |
| 9 | 98 | HPMCAS | 3000 | 1140 | 97,500 | 1,390,000 |
| 10 | 50 | CMEC | 3000 | 1440 | 125,100 | 1,730,000 |
| 11 | 50 | HPMC | 3000 | 1200 | 102,900 | 1,400,000 |
| Control 1 | — | — | 3000 | 112 | 9200 | 132,000 |
| (Crystalline | | | | | | |
| Form I of | | | | | | |
| Compound | | | | | | |
| A) | | | | | | |
| Example 1 | — | — | 3000 | 1150 | 75,900 | 233,000 |
| (Amorphous | | | | | | |
| Compound | | | | | | |
| A) | | | | | | |

The solid amorphous dispersions provided concentration-enhancement over that of crystalline Form I of Compound A alone (Control 1) and over amorphous Compound A alone (Example 1). The AUC₉₀ values for the dispersions of the invention are from 10.4- to 14.6-fold that of the crystalline control, and from 1.3- to 1.8-fold that of the amorphous Compound A alone (Example 1). The AUC₁₂₀₀ values for the dispersions of the invention are from 9.7- to 14.4-fold that of the crystalline control, and from 5.5- to 8.2-fold that of the amorphous Compound A alone (Example 1). This demonstrates that the addition of the concentration-enhancing polymer sustains the high dissolved drug concentration for an extended period of time relative to the amorphous Compound A alone.

### Example 13

The solid amorphous dispersion of Example 7 was analyzed using differential scanning calorimetry (DSC) to determine the amorphous character of Compound A in the dispersion. Sample pans were crimped at ambient conditions, and loaded into the furnace of a Perkin-Elmer Pyris 1 DSC with a robotic arm. The samples were heated at 2° C./min up to about 200° C. The glass transition temperatures of the samples were determined from the DSC scans. Crystalline Compound A (Control 1) and amorphous Compound A alone (Example 1) were also analyzed using the same procedure for comparison. The results are shown below in Table 10.

TABLE 10

| | Glass Transition | Crystalline Melt |
|---|---|---|
| Sample | Temperature (T_{g}) | Temperature (Tₘ) |
| Example 7 | 97.5 | — |
| Control 1 | — | 178.1 |
| (Crystalline Form I Compound A) | | |
| Example 1 | 99.1 | — |
| (Amorphous Compound A) | | |

Table 10 shows the glass transition temperature (T_{g}) of the amorphous Compound A alone (99.1° C.), and the sharp melting peak (Tₘ) of the crystalline drug (178.1° C.). The solid amorphous dispersion (Example 7) shows a T_{g} (97.5° C.) that is similar to the amorphous drug, and no melting peak, indicating a physical state distinct from that of crystalline Form I of Compound A alone.

### Example 14

Example 3 was examined using powder x-ray diffraction with a Bruker AXS D8 Advance diffractometer to determine the amorphous character of Compound A in the dispersion. Samples (approximately 100 mg) were packed in Lucite sample cups fitted with Si(511) plates as the bottom of the cup to give no background signal. Samples were spun in the φ plane at a rate of 30 rpm to minimize crystal orientation effects. The x-ray source (KCu_{α}, λ=1.54 Å) was operated at a voltage of 45 kV and a current of 40 mA. Data for each sample were collected over a period of 27 minutes in continuous detector scan mode at a scan speed of 1.8 seconds/step and a step size of 0.04°/step. Diffractograms were collected over the 2θ range of 4° to 30°. Crystalline Form I of Compound A (Control 1) was also analyzed using the same procedure for comparison. The results are shown in FIG. 1. The baselines of the respective patterns shown in FIG. 1 have been shifted relative to each other to allow the patterns to be viewed separately in the same figure. Example 3 exhibited a diffraction pattern showing only an amorphous halo, while Control 1 exhibited a pattern showing sharp peaks characteristic of crystalline drug. These data indicate that Compound A in the solid amorphous dispersion of Example 3 is amorphous and not crystalline.

### Example 15

These tests demonstrate the physical stability of a solid amorphous dispersion after storage under controlled temperature and humidity conditions to accelerate aging of the dispersion. PXRD diffractograms of Example 3 and Control 1 were measured before and after storage for 6 weeks under the following temperature and relative humidity (RH) conditions: 40° C./0% RH, 40° C./25% RH, 40° C./50% RH, or 40° C./75% RH. The results are shown in FIG. 2.

The results in FIG. 2 show that after storage under accelerated aging conditions for 6 weeks, Example 3 continued to exhibit a diffraction pattern with only an amorphous halo, and no sharp peaks characteristic of crystalline drug. The results demonstrate that the dispersion of Example 3 is physically stable under these conditions.

### Example 16

These tests further demonstrate the physical stability of the solid amorphous dispersions of Compound A after storage under controlled temperature and humidity conditions. The dissolution performance of the solid amorphous dispersion of Example 3 was measured before and after storage for 12 weeks under the following conditions: 30° C./60% RH, 40° C./25% RH, or 40° C./75% RH. Dissolution tests were performed as described in Example 1. The results are shown in Table 11.

TABLE 11

| | | Compound A | |
|---|---|---|---|
| | Time | Concentration | AUC |
| Storage Condition | (min) | (μg/mL) | (min * μg/mL) |
| Initial | 0 | 0 | 0 |
| | 4 | 872 | 1700 |
| | 10 | 928 | 7100 |
| | 20 | 1000 | 16800 |
| | 40 | 1050 | 37,300 |
| | 90 | 1070 | 90,200 |
| 30° C./60% RH | 0 | 0 | 0 |
| 12 weeks | 4 | 957 | 1900 |
| | 10 | 1070 | 8000 |
| | 20 | 1050 | 18,600 |
| | 40 | 1080 | 39,900 |
| | 90 | 11001 | 94,500 |
| 40° C./25% RH | 0 | 0 | 0 |
| 12 weeks | 4 | 902 | 1800 |
| | 10 | 969 | 7400 |
| | 20 | 1020 | 17,400 |
| | 40 | 1050 | 38,100 |
| | 90 | 1030 | 90,000 |
| 40° C./75% RH | 0 | 0 | 0 |
| 12 weeks | 4 | 897 | 1800 |
| | 10 | 880 | 7100 |
| | 20 | 976 | 16,400 |
| | 40 | 994 | 36,100 |
| | 90 | 1030 | 86,800 |

The concentrations of Compound A obtained in these samples were used to determine the maximum concentration of drug, MDC₉₀, and the area under the concentration-versus-time curve, AUC₉₀, during the initial ninety minutes. The results are shown in Table 12.

TABLE 12

| | MDC₉₀ | AUC₉₀ |
|---|---|---|
| Storage Condition | (μg/mL) | (min * μg/mL) |
| Initial | 1070 | 90,200 |
| 30° C./60% RH | 1100 | 94,500 |
| 40° C./25% RH | 1050 | 90,000 |
| 40° C./75% RH | 1030 | 86,800 |

As can be seen from the data, the AUC₉₀ for the dispersion remained relatively constant after 12 weeks storage under accelerated aging conditions. This demonstrates that the dispersion of Example 3 is physically stable under these conditions.

### Example 17

The solid amorphous dispersion of Example 17 was formed using a “mini” spray-drying apparatus, as described for Examples 4-11. The spray solution consisted of 2500 mg Compound A, 247.5 mg HPMCAS (AQUOT-MG), and 2.5 mg BHT, in 31 g methanol. The spray solution was pumped into the spray chamber at a rate of 1.3 mLs/min, and the inlet temperature was 80° C.

### Example 18

Compound A dispersions were formulated with the antioxidant butylated hydroxytoluene (BHT), or stored with an oxygen absorbing packet, to improve chemical stability of Compound A.

The dispersion of Example 3, the dispersion of Example 17, and the dispersion of Example 3 in a closed container with an oxygen absorber with the trade name Fresh Pax™ (available from Multisorb Technology), were stored at 40° C./75% RH. Samples were analyzed for Compound A degradation products after 10 days, 3 weeks, or 6 weeks, using HPLC to determine the amount of degradant present in the sample. The results are shown below in Table 13.

TABLE 13

Degradant
(%)

| | | | Example 17 | Example 3 |
|---|---|---|---|---|
| | | Example 3 | 40° C./75% | 40° C./75% |
| Storage | | 40° C./75% | RH, open | RH, closed |
| Time | | RH open | added BHT | O₂ scavenger |
| 0 | | 0.09 | 0.09 | 0.09 |
| 10 | days | 0.35 | 0.09 | 0.09 |
| 3 | weeks | 0.53 | 0.15 | 0.09 |
| 6 | weeks | 0.98 | 0.27 | 0.10 |

The dispersions showed reduced degradation of Compound A for samples stored with BHT or an O₂ absorber.

### Examples 19-34

Solid amorphous dispersions comprising Compound A, the concentration-enhancing polymer HPMCAS, and a stabilizing agent were formulated to improve chemical stability. The solid amorphous dispersions of Examples 19-33 were prepared with different types and amounts of bases, different amounts of the antioxidant BHT, or both base and antioxidant. Base was used to neutralize a portion of the polymer for the solid amorphous dispersions of Examples 19-28 and 30-33. Example 34 was prepared without a stabilizing agent for comparison.

The dispersions of Examples 19-22, 24-29, 32, and 33 were formed using a “mini” spray-drying apparatus, as described for Examples 4-11. The composition of each formulation is shown in Table 14. Spray solutions each contained 10 wt % solids in a solution of 20/80 water/methanol (wt/wt). The spray solutions were pumped into the spray chamber at a rate of 1.3 mL/min, and the inlet temperature was 74° C., for all of the dispersions.

The dispersions of Examples 23, 30, 31, and 34 were formed using a Niro PSD-1, as described for Example 3. The composition of each formulation is shown in Table 14. Spray solutions each contained 10% solids in a solution of 20/80 water/methanol (wt/wt).

TABLE 14

| Example | Drug Mass | Polymer | | Base Mass | BHT Mass |
|---|---|---|---|---|---|
| No. | (mg) | Mass (mg) | Base | (mg) | (mg) |
| 19 | 1125 | 123 | Ca(OAc)₂ | 2 | — |
| 20 | 1125 | 115 | Ca(OAc)₂ | 10 | — |
| 21 | 1125 | 122 | Ca(OAc)₂ | 2 | 1 |
| 22 | 1125 | 104 | Ca(OAc)₂ | 20 | 1 |
| 23 | 85,500 | 7,600 | Ca(OAc)₂ | 1,900 | — |
| 24 | 1125 | 123 | Ca₃citrate₂ | 2 | — |
| 25 | 1125 | 105 | K(OAc) | 20 | — |
| 26 | 1125 | 125 | CaCO₃ | 3.4 | — |
| 27 | 1125 | 125 | CaCO₃ | 6.5 | — |
| 28 | 1125 | 125 | Ca(OH)₂ | 5.6 | — |
| 29 | 900 | 124 | — | — | 1 |
| 30 | 85,500 | 9,405 | Ca(OH)₂ | 192 | 95 |
| 31 | 85,500 | 9,500 | Ca(OH)₂ | 368 | — |
| 32 | 1125 | 123 | Ca(OH)₂ | 2.5 | 2 |
| 33 | 1125 | 120 | Ca(OH)₂ | 2.4 | 5 |
| 34 | 85,500 | 9,500 | — | — | — |

The dispersions of Examples 19-34 were aged in a loosely foil-covered HPLC vial at 40° C. and 75% relative humidity (RH).

Samples were analyzed for degradation products after 6 weeks using HPLC. The results are shown below in Table 15.

TABLE 15

| | | | Degradant after 6 |
|---|---|---|---|
| Example No. | Base | BHT | weeks (wt %) |
| 19 | Ca(OAc)₂ | — | 0.17 |
| 20 | Ca(OAc)₂ | — | 0.09 |
| 21 | Ca(OAc)₂ | 0.1% | 0.08 |
| 22 | Ca(OAc)₂ | 0.1% | 0.07 |
| 23 | Ca(OAc)₂ | — | 0.45 |
| 24 | Ca₃citrate₂ | — | 0.29 |
| 25 | K(OAc) | — | 0.12 |
| 26 | CaCO₃ | — | 0.33 |
| 27 | CaCO₃ | — | 0.15 |
| 28 | Ca(OH)₂ | — | 0.12 |
| 29 | — | 0.1% | 0.12 |
| 30 | Ca(OH)₂ | 0.1% | 0.39 |
| 31 | Ca(OH)₂ | — | 0.37 |
| 32 | Ca(OH)₂ | 0.2% | 0.10 |
| 33 | Ca(OH)₂ | 0.5% | 0.09 |
| 34 | — | — | 0.76 |

The results show that addition of base, an antioxidant, or both, improves the chemical stability of Compound A in solid amorphous dispersions comprising HPMCAS.

### Example 35

The chemical stability of a physical mixture of Compound A and HPMCAS was evaluated. Amorphous Compound A was prepared using a mini-spray drier as described in Examples 4-11. The spray solution, containing 1.5 wt % solids in methanol/water (8/2, vol./vol.), was sprayed at a rate of 1.3 mL/min, with an inlet temperature of 70° C. and an outlet temperature at ambient conditions.

HPMCAS was prepared using a PSD-1 spray drier as described in Example 3. The spray solution, containing 12 wt % solids in acetone, was sprayed at a rate of 183-215 g/min, with an inlet temperature of 100° C. and an outlet temperature of 37° C.

Compound A and HPMCAS were mixed together in a 9:1 ratio and stored under various temperature and relative humidity conditions for chemical stability testing, with results in the Table 16 below.

TABLE 16

| | Degradation Products (%) After Aging | | |
|---|---|---|---|
| Time | 5° C., closed | 30° C./60% RH | 40° C./75% RH |
| 3 weeks | 0.02* | 0.02* | 0.002* |
| 6 weeks | 0** | 0.07 | 0.07 |

*Amount below quantitation limit.
**Amount below detection limit.

### Example 36

In vivo dissolution tests were performed using dogs to demonstrate that the amorphous dispersions of the invention provide concentration-enhancement of Compound A. The solid amorphous dispersion of Example 3 was dosed to a group of 6 fasted beagle dogs and drug release was monitored by periodically withdrawing blood and measuring the plasma drug concentration. The area under the concentration-versus-time curve (“AUC_{O-Tlast}”, ng*hr/mL), from the time the dose was administered to the last sample, is shown in Table 17. Crystalline Form I of Compound A (Control 1) and amorphous Compound A (Example 1) were tested using a similar protocol.

The dose was administered to dogs as a suspension in a solution containing 0.5 wt % Methocel® (HPMC, USP grade, 4000 cps, Dow Chemical Co.). Oral administration of the aqueous drug suspensions was facilitated using an oral gavage equipped with a polyethylene tube insert. The polyethylene tube insert was used to accurately deliver the desired volume of dose by displacement, without the need for additional volume of water to rinse the tube. The dose was 25 mgA/kg (“mgA” refers to mg of active drug).

TABLE 17

| | AUC_{0-Tlast} |
|---|---|
| Dosage Form | (ng*hr/mL) |
| Control 1 (Crystalline Form I of | 300 |
| Compound A) | |
| Example 1 (Amorphous Compound | 3500 |
| A) | |
| Example 3 | 13,400 |

The above results clearly indicate that both amorphous Compound A alone (Example 1) and the solid amorphous dispersion of Compound A and a concentration-enhancing polymer (Example 3) provide higher drug concentrations in vivo than crystalline Form I of Compound A alone. The relative bioavailability (AUC of the test composition divided by AUC of crystalline Form I of Compound A alone) for amorphous Compound A of Example 1 was 11.7-fold that of crystalline Form I of Compound A alone, while the relative bioavailability of the solid amorphous dispersion of Example 3 was 44.7-fold that of crystalline Form I of Compound A alone.

### Example 37

This example shows that even small amounts of concentration-enhancing polymer combined with amorphous Compound A sustain the concentration of dissolved Compound A in an in vitro use environment. Examples 37A, 37B and 37C were prepared as in Example 2, but with the following exceptions: Example 37A was 5.4 mg Compound A and 0.6 mg HPMCAS; Example 37B was 5.4 mg Compound A and 0.167 mg HPMCAS; and Example 37C was 5.4 mg Compound A and 0.055 mg HPMCAS. Example 37D consisted of amorphous Compound A alone. Dissolution tests were performed as in Example 2, with the results summarized in Table 18.

TABLE 18

| | Compound A | MDC₃₆₀ | AUC₃₆₀ | C₁₂₀₀ |
|---|---|---|---|---|
| Example | (wt %) | μg/ml | min*μg/ml | μg/ml |
| 37A | 90 | 1360 | 458,000 | 1200 |
| 37B | 97 | 1350 | 432,000 | 1350 |
| 37C | 99 | 1280 | 436,000 | 1300 |
| 37D | 100 | 1190 | 180,000 | 140 |

MDC₃₆₀ is the maximum dissolved drug concentration within the first 360 minutes, and AUC₃₆₀ is the area under the dissolved drug concentration versus time curve at 360 minutes. The results show that combining even small amounts of polymer with the amorphous Compound A sustains the dissolved drug concentration in an aqueous use environment relative to amorphous Compound Alone.

In the examples described below, unless otherwise indicated, all temperatures in the following description are in degrees Celsius (° C.) and all parts and percentages are by weight, unless indicated otherwise.

Various starting materials and other reagents were purchased from commercial suppliers, such as Aldrich Chemical Company or Lancaster Synthesis Ltd., and used without further purification, unless otherwise indicated.

The reactions set forth below were performed under a positive pressure of nitrogen, argon or with a drying tube, at ambient temperature (unless otherwise stated), in anhydrous solvents. Analytical thin-layer chromatography was performed on glass-backed silica gel 60° F. 254 plates (Analtech (0.25 mm)) and eluted with the appropriate solvent ratios (v/v). The reactions were assayed by high-pressure liquid chromotagraphy (HPLC) or thin-layer chromatography (TLC) and terminated as judged by the consumption of starting material. The TLC plates were visualized by UV, phosphomolybdic acid stain, or iodine stain. ¹H-NMR spectra were recorded on a Bruker instrument operating at 300 MHz and ¹³C-NMR spectra were recorded at 75 MHz. NMR spectra are obtained as DMSO-d₆ or CDCl₃ solutions (reported in ppm), using chloroform as the reference standard (7.25 ppm and 77.00 ppm) or DMSO-d₆ ((2.50 ppm and 39.52 ppm)). Other NMR solvents were used as needed. When peak multiplicities are reported, the following abbreviations are used: s=singlet, d=doublet, t=triplet, m=multiplet, br=broadened, dd=doublet of doublets, dt=doublet of triplets. Coupling constants, when given, are reported in Hertz. Infrared spectra were recorded on a Perkin-Elmer FT-IR Spectrometer as neat oils, as KBr pellets, or as CDCl₃ solutions, and when reported are in wave numbers (cm⁻¹). The mass spectra were obtained using LC/MS or APCI. All melting points are uncorrected. All final products had greater than 95% purity (by HPLC at wavelengths of 220 nm and 254 nm).

### Preparation of Compound A

In the following examples and preparations, “Et” means ethyl, “Ac” means acetyl, “Me” means methyl, “Ph” means phenyl, (PhO)₂POCI means chlorodiphenylphosphate, “HCl” means hydrochloric acid, “EtOAc” means ethyl acetate, “Na₂CO₃” means sodium carbonate, “NaOH” means sodium hydroxide, “NaCl” means sodium chloride, “NEt₃” means triethylamine, “THF” means tetrahydrofuran, “DIC” means diisopropylcarbodiimide, “HOBt” means hydroxy benzotriazole, “H₂O” means water, “NaHCO₃” means sodium hydrogen carbonate, “K₂CO₃” means potassium carbonate, “MeOH” means methanol, “i-PrOAc” means isopropyl acetate, “MgSO₄” means magnesium sulfate, “DMSO” means dimethylsulfoxide, “AcCl” means acetyl chloride, “CH₂Cl₂” means methylene chloride, “MTBE” means methyl t-butyl ether, “DMF” means dimethyl formamide, “SOCl₂” means thionyl chloride, “H₃PO₄” means phosphoric acid, “CH₃SO₃H” means methanesulfonic acid, “AC₂O” means acetic anhydride, “CH₃CN” means acetonitrile, and “KOH” means potassium hydroxide.

### Example 38

### Preparation of (4R)-4-allylcarbamoyl-5,5-dimethyl-thiazolidine-3-carboxylic Acid Tert-Butyl Ester

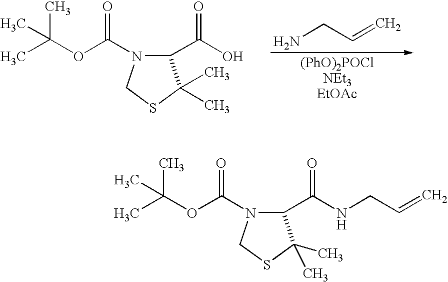

(4R)-5,5-Dimethyl-thiazolidine-3,4-dicarboxylic acid 3-tert-butyl ester (which can be prepared according to the methods of Ikunaka, M. et al., Tetrahedron Asymm. 2002, 13, 1201; Mimoto, T. et al., J. Med. Chem. 1999, 42, 1789; and Mimoto, T. et al., European Patent Application 0574135A1 (1993), 250 g; 0.957 mol) was added to an argon-purged 5-L flask and was dissolved in EtOAc (1.25 L). The solution was cooled to 2° C. and (PhO)₂POCI (208 mL; 1.00 mol) was then added in one portion. NEt₃ (280 mL; 2.01 mol) was added dropwise via addition funnel and the resulting suspension was then stirred at 0° C. Seven minutes later, allylamine (75.4 mL; 1.00 mol) was added dropwise. The ice bath was removed and the suspension was allowed to warm to room temperature. One-half hour later, 1 N HCl (750 mL; 0.750 mol) was added. The mixture was transferred to a 4-L separatory funnel using EtOAc (50 mL) for rinsing. The layers were separated. The organic fraction was washed with 7.2% aqueous Na₂CO₃ (2×1.25 L), and was then transferred to a 3-L distillation flask and was diluted with EtOAc (400 mL). The solution was dried azeotropically and concentrated to a volume of 800 mL by distillation of EtOAc at one atmosphere. After cooling to 25° C., the resulting clear yellowish EtOAc solution of (4R)-4-allylcarbamoyl-5,5-dimethyl-thiazolidine-3-carboxylic acid tert-butyl ester was carried on directly into the next step. An aliquot was removed and concentrated to give (4R)-4-allylcarbamoyl-5,5-dimethyl-thiazolidine-3-carboxylic acid tert-butyl ester as a white crystalline solid: mp=94-98° C., ¹H NMR (300 MHz, CDCl₃) δ 6.12 (br s, 1H), 5.88 (app ddt, J=10.2, 17.1, 5.6 Hz, 1H), 5.28 (app dq, J=17.1, 1.5 Hz, 1H), 5.18 (app dd, J=1.2, 10.2 Hz, 1H), 4.68 (s, 2H), 4.14 (br s, 1H), 3.95 (br t, J=5.4 Hz, 2H), 1.62 (s, 3H), 1.49 (s, 9H), 1.46 (s, 3H); ¹³C NMR (75 MHz, CDCl₃) δ 170.0, 154.0, 134.4, 116.9, 82.0, 73.3, 54.0, 48.7, 42.0, 30.6, 28.6, 24.6; MS (Cl) m/z 301.1599 (301.1586 calcd for C₁₄H₂₅N₂O₃S, M+H⁺); elemental analysis calcd for C₁₄H₂₄N₂O₃S: C, 55.97; H, 8.05; N, 9.32; found: C, 56.11; H, 8.01; N, 9.11.

### Example 39

### Preparation of (4R)-5,5-dimethyl-thiazolidine-4-carboxylic Acid Allylamide

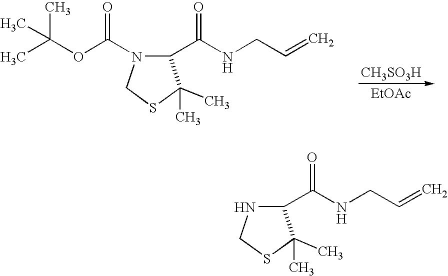

Methanesulfonic acid (155 mL; 2.39 mol) was added dropwise to the EtOAc solution of (4R)-4-allylcarbamoyl-5,5-dimethyl-thiazolidine-3-carboxylic acid tert-butyl ester in a 3-L flask. After stirring at room temperature overnight, the solution was cooled to 7° C. and H₂O (400 mL) was poured in. The mixture was transferred to a 4-L separatory funnel [using H₂O (30 mL) for rinsing] and the layers were separated. The organic fraction was extracted with H₂O (190 mL). The combined H₂O extracts were transferred to a 5-L flask and were cooled to 8° C. The pH was adjusted from 0.4 to 9.3 using 3 N NaOH (˜1.05 L). 2-Methyltetrahydrofuran (1.55 L) was poured in, followed by the addition of NaCl (150 g). The ice bath was removed and the mixture was allowed to warm to room temperature. The pH was readjusted to 9.0 using 3 N NaOH (˜1 mL). The mixture was transferred to a 4-L separatory funnel, using 2-methyltetrahydrofuran (50 mL) for rinsing, and the layers were separated. The aqueous phase was extracted with 2-methyltetrahydrofuran (950 mL). The organic extracts were vacuum-filtered through Celite directly into a 5-L distillation flask, using 2-methyltetrahydrofuran (200 mL) for rinsing. The solution was dried azeotropically and concentrated to a volume of 1.2 L by distillation of 2-methyltetrahydrofuran at one atmosphere. A measured aliquot was concentrated and weighed, which showed that 161 g of (4R)-5,5-Dimethyl-thiazolidine-4-carboxylic acid allylamide was present in solution [84% from (4R)-5,5-dimethyl-thiazolidine-3,4-dicarboxylic acid 3-tert-butyl ester]. This solution was then carried on directly into the next step. The concentrated aliquot from above yielded (4R)-5,5-Dimethyl-thiazolidine-4-carboxylic acid allylamide as a crystalline solid: mp=45-47° C., ¹H NMR (300 MHz, CDCl₃) δ 6.73 (br s, 1H), 5.87 (app ddt, J=10.2, 17.1, 5.7 Hz, 1H), 5.17-5.27 (m, 2H), 4.27 (AB q, J_{AB}=9.7 Hz, Δv=22.5 Hz, 2H), 2.94 (app tt, J=1.5, 5.8 Hz, 2H), 3.51 (s, 1H), 1.74 (s, 3H), 1.38 (s, 3H); ¹³C NMR (75 MHz, CDCl₃) δ 169.7, 134.4, 116.9, 74.8, 57.2, 51.6, 41.9, 29.1, 27.3; MS (Cl) m/z 201.1063 (201.1062 calcd for C₉H₁₇N₂OS, M+H⁺); elemental analysis calcd for C₉H₁₅N₂OS: C, 53.97; H, 8.05; N, 13.99; found: C, 53.93; H, 8.09; N, 14.07.

### Example 40

### Preparation of (2S,3S)-3-(3-acetoxy-2-methyl-benzoylamino)-2-hydroxy-4-phenyl-butyric Acid

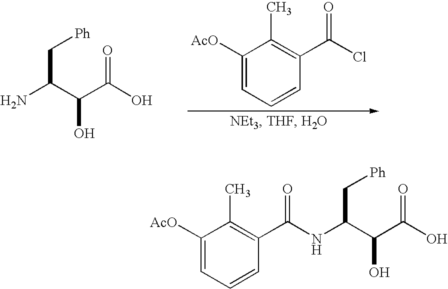

(2S,3S)-3-Amino-2-hydroxy-4-phenyl-butyric acid (which can be prepared according to the method of Pedrosa et al., Tetrahedron Asymm. 2001, 12, 347; M. Shibasaki et al., Tetrahedron Lett. 1994, 35, 6123; and Ikunaka, M. et al. Tetrahedron Asymm. 2002, 13, 1201; 185 g; 948 mmol) was added to a 5-L flask and was suspended in THF (695 mL). H₂O (695 mL) was poured in, followed by NEt₃ (277 mL; 1990 mmol). After stirring for 45 min, the solution was cooled to 6° C. A solution of acetic acid 3-chlorocarbonyl-2-methyl-phenyl ester (201 g; 948 mmol) in THF (350 mL) was then added dropwise. One-half hour later, the pH was adjusted from 8.7 to 2.5 with 6 N HCl (−170 mL). Solid NaCl (46 g) was added, the ice bath was then removed and the mixture was stirred vigorously while warming to room temperature. The mixture was transferred to 4-L separatory funnel, using 1:1 THF/H₂O (50 mL) for the transfer, and the lower aqueous phase was then removed. The organic fraction was transferred to a 5-L distillation flask, and was then diluted with fresh THF (2.5 L). The solution was azeotropically dried and concentrated to a volume of 1.3 L by distillation of THF at one atmosphere. To complete the azeotropic drying, fresh THF (2.0 L) was added and the solution was concentrated to 1.85 L by distillation at one atmosphere and was then held at 55° C. n-Heptane (230 mL) was added dropwise via addition funnel and the solution was then immediately seeded. After crystallization had initiated, additional n-heptane (95 mL) was added dropwise. The resulting crystal slurry was stirred vigorously for 7 min. Additional n-heptane (1.52 L) was then added as a slow stream. The crystal slurry was then allowed to cool to room temperature slowly and stir overnight. The suspension was vacuum-filtered and the filter cake was then washed with 1:1 THF/n-heptane (700 mL). After drying in a vacuum oven at 45-50° C., 324 g (92%) of (2S,3S)-3-(3-acetoxy-2-methyl-benzoylamino)-2-hydroxy-4-phenyl-butyric acid was obtained as a crystalline solid contaminated with ˜7 mol % Et₃N·HCl: mp=189-191° C., ¹H NMR (300 MHz, DMSO-d₆) δ 12.65 (br s, 1H), 3.80 (d, J=9.7 Hz, 1H), 7.16-7.30 (m, 6H), 7.07 (dd, J=1.1, 8.0 Hz, 1H), 7.00 (dd, J=1.1, 7.5 Hz), 4.40-4.52 (m, 1H), 4.09 (d, J=6.0 Hz, 1H), 2.92 (app dd, J=2.9, 13.9 Hz, 1H), 2.76 (app dd, J=11.4, 13.9 Hz, 1H), 2.29 (s, 3H), 1.80 (s, 3H); ¹³C NMR (75 MHz, DMSO-d₆) δ 174.4, 169.3, 168.1, 149.5, 139.7, 139.4, 129.5, 128.3, 127.9, 126.5, 126.3, 124.8, 123.3, 73.2, 53.5, 35.4, 20.8, 12.6; MS (Cl) m/z 372.1464 (372.1447 calcd for C₂₀H₂₂NO₆, M+H+); elemental analysis calcd for C₂₀H₂₁NO₆•0.07 Et₃N•HCl: C, 64.34; H, 5.86; N, 3.95; Cl, 0.70; found: C, 64.27; H, 5.79; N, 3.96; Cl; 0.86.

### Example 41

### Preparation of Acetic Acid 3-{(1S,2S)-3-[(4R)-4-allylcarbamoyl-5,5-dimethyl-thiazolidin-3-yl]-1-benzyl-2-hydroxy-3-oxo-propylcarbamoyl}-2-methyl-phenyl Ester

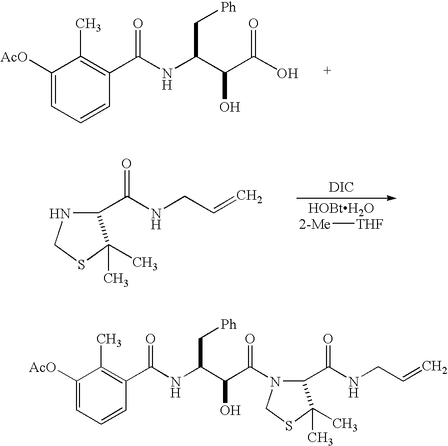

(2S,3S)-3-(3-Acetoxy-2-methyl-benzoylamino)-2-hydroxy-4-phenyl-butyric acid (271 g; 731 mmol) was added to a 5-L flask containing a solution of (4R)-5,5-Dimethyl-thiazolidine-4-carboxylic acid allylamide (161 g; 804 mmol) in 2-methyltetrahydrofuran (1.20 L total solution), while using 2-methyltetrahydrofuran (500 mL) for rinsing. HOBt•H₂O (32.6 g; 241 mmol) was added, using 2-methyltetrahydrofuran (50 mL) for rinsing. The white suspension was allowed to stir at room temperature for 10 min. Diisopropylcarbodiimide (119 mL; 760 mmol) was added in three portions (40 mL+40 mL+39 mL) at 30 min intervals. One hour after the final DIC addition, Celite (100 g) was added and the suspension was allowed to stir at room temperature for 3 h. The mixture was vacuum-filtered, while 2-methyltetrahydrofuran (400 mL) was used to rinse over the solids and wash the resulting filter cake. The filtrate was transferred to 4-L separatory funnel, using 2-methyltetrahydrofuran (50 mL) for rinsing. The solution was washed with 1 N HCl (1.25 L), and then with an aqueous solution of NaHCO₃ (27 g), NaCl (134 g) and H₂O (1.25 L). The resulting organic phase was transferred to a 3-L distillation flask and the solution was then reduced to a volume of 1.12 L by distillation of 2-methyltetrahydrofuran at one atmosphere. The solution was then diluted with 2-methyltetrahydrofuran (230 mL) to bring the total volume to 1.35 L. After cooling the solution to 23° C., the solution of crude acetic acid 3-{(1S,2S)-3-[(4R)-4-allylcarbamoyl-5,5-dimethyl-thiazolidin-3-yl]-1-benzyl-2-hydroxy-3-oxo-propylcarbamoyl}-2-methyl-phenyl ester on directly into the next step.

### Example 42

### Preparation of (4R)-3-[(2S,3S)-2-hydroxy-3-(3-hydroxy-2-methyl-benzoylamino)-4-phenyl-butyryl]-5,5-dimethyl-thiazolidine-4-carboxylic Acid Allylamide

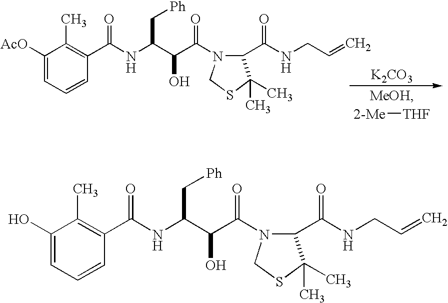

MeOH (330 mL) and K₂CO₃ (66.9 g; 484 mmol) were sequentially added to a 2-methyltetrahydrofuran solution of crude acetic acid 3-{(1S,2S)-3-[(4R)-4-allylcarbamoyl-5,5-dimethyl-thiazolidin-3-yl]-1-benzyl-2-hydroxy-3-oxo-propylcarbamoyl}-2-methyl-phenyl ester (theoretical amount: 405 g; 731 mmol) in a 3-L flask at room temperature. Two and a half hours later, additional K₂CO₃ (20 g; 144 mmol) was added. Three hours later the reaction mixture was vacuum-filtered on a pad of Celite, using 4:1 2-methyltetrahydrofuran/MeOH (330 mL) for rinsing over the solids and washing the filter cake. The filtrate was transferred to a 6-L separatory funnel, using 4:1 2-methyltetrahydrofuran/MeOH (80 mL) for rinsing. The solution was diluted with i-PrOAc (1.66 L) and was then washed with a solution of NaCl (83.0 g) in H₂O (1.60 L). The organic fraction was washed with 0.5 N HCl (1.66 L) and then with a saturated aqueous NaCl solution (400 mL). The resulting organic fraction was transferred to a 4-L Erlenmeyer flask and MgSO₄ (120 g) was added. After stirring for 10 min, the mixture was vacuum-filtered directly into a 5-L distillation flask, using 2:1 i-PrOAc/2-methyltetrahydrofuran (600 mL) for rinsing the separatory funnel and Erlenmeyer flask and washing the MgSO₄. The 2-methyltetrahydrofuran was displaced by distillation at one atmosphere with the simultaneous addition of i-PrOAc in five portions (a total of 3.60 L was used), while maintaining a minimum pot volume of ˜2.50 L. The resulting crystallizing mixture was cooled to 75° C. and was held at this temperature for 30 min. The suspension was then allowed to slowly cool to room temperature overnight. The suspension was vacuum-filtered, using i-PrOAc (600 mL) for transferring and washing the crystals. After drying in a vacuum oven at 40° C., 204 g (54% from (2S,3S)-3-(3-Acetoxy-2-methyl-benzoylamino)-2-hydroxy-4-phenyl-butyric acid) of crystalline (4R)-3-[(2S,3S)-2-Hydroxy-3-(3-hydroxy-2-methyl-benzoylamino)-4-phenyl-butyryl]-5,5-dimethyl-thiazolidine-4-carboxylic acid allylamide was obtained. This material was recrystallized as described below.

### Example 43

### Recrystallization of (4R)-3-[(2S,3S)-2-hydroxy-3-(3-hydroxy-2-methyl-benzoylamino)-4-phenyl-butyryl]-5,5-dimethyl-thiazolidine-4-carboxylic Acid Allylamide

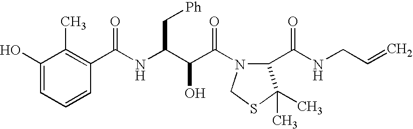

(4R)-3-[(2S,3S)-2-Hydroxy-3-(3-hydroxy-2-methyl-benzoylamino)-4-phenyl-butyryl]-5,5-dimethyl-thiazolidine-4-carboxylic acid allylamide (193 g, 378 mmol) was added to a 5-L flask and was then suspended in EtOAc (1.28 L). After heating the suspension to 76° C., MeOH (68 mL) was added and the internal temperature was then reduced to 70° C. n-Heptane (810 mL) was added dropwise to the solution, while maintaining the internal temperature at 70° C. After the n-heptane addition was complete, the resulting crystal suspension was held at 70° C. for 30 min, and was then allowed to slowly cool to room temperature overnight. The suspension was vacuum-filtered, using 1.6:1 EtOAc/n-heptane (500 mL) to transfer and wash the crystals. The crystals were then dried in a vacuum oven at 45° C. to give 162 g (84% recovery) of purified (4R)-3-[(2S,3S)-2-Hydroxy-3-(3-hydroxy-2-methyl-benzoylamino)-4-phenyl-butyryl]-5,5-dimethyl-thiazolidine-4-carboxylic acid allylamide as a white crystalline solid: mp=173-175° C., ¹H NMR (300 MHz, DMSO-d₆) displayed a 10:1 mixture of rotamers, major rotamer resonances δ 9.35 (s, 1H), 8.04-8.15 (m, 2H), 7.13-7.38 (m, 5H), 6.96 (t, J=7.7 Hz, 1H), 6.79 (d, J=7.2 Hz, 1H), 6.55 (d, J=7.5 Hz, 1H), 5.71-5.87 (m, 1H), 5.45 (br d, J=6.2 Hz, 1H), 4.98-5.27 (m, 4H), 4.38-4.52 (m, 3H), 3.58-3.86 (m, 2H), 2.68-2.90 (m, 2H), 1.84 (s, 3H), 1.52 (s, 3H), 1.37 (s, 3H) [characteristic minor rotamer resonances δ 9.36 (s), 8.21 (d, J=10.5 Hz), 7.82 (5, J=5.8 Hz), 4.89 (s), 4.78 (AB q, J_{AB}=9.8 Hz, Δv=27.1 Hz), 4.17-4.24 (m), 2.93-3.01 (m), 1.87 (s), 1.41 (s)]; ¹³C NMR (75 MHz, DMSO-d₆) displayed a ˜10:1 mixture of rotamers, major rotamer resonances δ 170.4, 169.5, 168.2, 155.7, 139.6, 139.4, 135.5, 135.4, 129.9, 128.2, 126.2, 126.1, 121.9, 117.8, 115.6, 72.4, 72.1, 53.1, 51.4, 48.2, 41.3, 34.2, 30.5, 25.0, 12.6 [characteristic minor rotamer resonances δ 171.4, 169.7, 168.6, 139.0, 129.5, 128.4, 70.6, 54.2, 49.1, 41.5, 31.4, 24.8]; MS (Cl) m/z 512.2224 (512.2219 calcd for C₂₇H₃₄N₃O₅S, M+H⁺), elemental analysis calcd for C₂₇H₃₃N₃O₅S: C, 63.38; H, 6.50; N, 8.22; found: C, 63.19; H, 6.52; N, 8.10.

### Example 44

### Preparation of (R)-5,5-dimethyl-thiazolidine-4-carboxylic Acid Allylamide; Hydrochloride

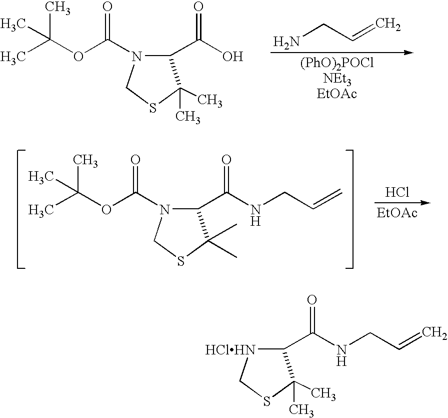

A solution of (R)-5,5-Dimethyl-thiazolidine-3,4-dicarboxylic acid 3-tert-butyl ester (105 kg, 402 mol) and ethyl acetate (690 L) was treated with diphenylchlorophosphate (113 kg, 422 mol) and was then cooled to 0° C. NEt₃ (85.5 kg, 844 mol) was added while maintaining the temperature at 5° C., and the mixture was then held at this temperature for 2 h. The mixture was cooled to 0° C., and allylamine (24.1 kg, 422 mol) was then added while maintaining the temperature at 5° C. The mixture was warmed to 20° C. and was then quenched with 10 wt. % aqueous HCl (310 L). After separation of the layers, the organic fraction was washed with 8.6 wt. % aqueous Na₂CO₃ (710 L). After separation of the layers, the aqueous fraction was extracted with ethyl acetate (315 L). The combined ethyl acetate extracts containing AG-074278 were dried by azeotropic distillation at one atmosphere, while maintaining a minimum pot volume of approximately 315 L. The resulting suspension of (R)-4-Allylcarbamoyl-5,5-dimethyl-thiazolidine-3-carboxylic acid tert-butyl ester was cooled to 5° C. A 13 wt. % solution of anhydrous HCl (36.8 kg, 1008 mol) in ethyl acetate (263 L) was cooled to 5° C. and was then added to the (R)-4-Allylcarbamoyl-5,5-dimethyl-thiazolidine-3-carboxylic acid tert-butyl ester suspension while maintaining the temperature at 15° C. The resulting suspension was held at 20° C. for 19 h, and was then cooled and held at 5° C. for 2 h. The suspension was then filtered, using cold ethyl acetate for rinsing. The wet cake was dried under vacuum at 45° C. to give 90.5 kg (95.2%) of (R)-5,5-Dimethyl-thiazolidine-4-carboxylic acid allylamide hydrochloride as a white solid: ¹H NMR (300 MHz, DMSO-d₆) δ 8.94 (app t, J=5.5 Hz, 1H), 5.82 (ddt, J=10.4, 17.2, 5.2 Hz, 1H), 5.19-5.25 (m, 1H), 5.10-5.14 (m, 1H), 4.38 (AB q, J_{AB}=9.8 Hz, Δv=14.5 Hz, 2H), 4.08 (s, 1H), 3.72-3.91 (m, 2H), 1.58 (s, 3H), 1.32 (s, 3H); ¹³C NMR (75 MHz, DMSO-d₆) δ 161.7, 132.2, 114.0, 67.9, 51.4, 43.5, 39.3, 25.3, 24.3; MS (Cl) m/z 201.1070 (201.1062 calcd for C₉H₁₇N₂OS, M+H+); elemental analysis calcd for C₉H₁₇ClN₂OS: C, 45.65; H, 7.24; N, 11.83; Cl, 14.97; found: C, 45.41; H, 7.33; N, 11.69; Cl, 15.22.

### Example 45

### Preparation of (2S,3S)-2-acetoxy-3-(3-acetoxy-2-methyl-benzoylamino)-4-phenyl-butyric Acid

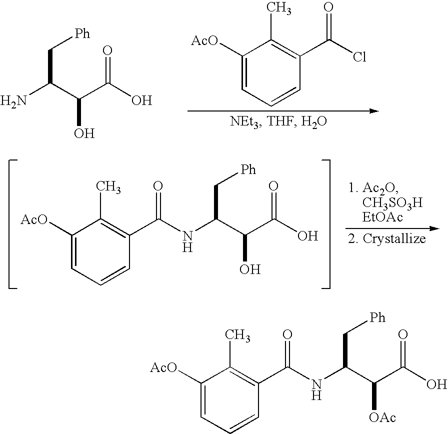

A mixture of (2S,3S)-3-Amino-2-hydroxy-4-phenyl-butyric acid (110 kg, 563 mol), NaCl (195 kg), and THF (413 L) was charged with NEt₃ (120 kg, 1183 mol) and H₂O (414 L) at ambient temperature. The resulting mixture was cooled to 0° C. Acetic acid 3-chlorocarbonyl-2-methyl-phenyl ester (120 kg, 563 mol) was added to a separate reactor and was then dissolved in THF (185 L). The resulting solution of acetic acid 3-chlorocarbonyl-2-methyl-phenyl ester was cooled to 10° C., and was then added to the (2S,3S)-3-amino-2-hydroxy-4-phenyl-butyric acid mixture while maintaining the temperature<10° C. during addition. The resulting biphasic mixture was agitated at 5° C. for 1 h, and was then adjusted to pH 2.5-3.0 with concentrated HCl (62 kg). The mixture was then warmed to 25° C., and the layers were separated. The resulting THF fraction, containing (2S,3S)-3-(3-acetoxy-2-methyl-benzoylamino)-2-hydroxy-4-phenyl-butyric acid, was partially concentrated by distillation at one atmosphere. THF was then replaced with ethyl acetate by distillation at one atmosphere, while maintaining a minimum pot volume of 1500 L. The resulting solution was cooled to 25° C., and was then charged with acetic anhydride (74.8 kg, 733 mol) and methanesulfonic acid (10.8 kg, 112 mol). The mixture was heated at 70° C. for approximately 3 h. The mixture was cooled to 25° C., and was then quenched with H₂O (1320 L) while maintaining the temperature at 20° C. After removal of the aqueous layer, the organic fraction was charged with ethyl acetate (658 L) and H₂O (563 L). After agitation, the aqueous phase was removed. The organic fraction was washed twice with 13 wt. % aqueous NaCl (2×650 L). The organic fraction was partially concentrated and dried by vacuum distillation (70-140 mm Hg) to a volume of approximately 1500 L. The resulting solution was heated to 40° C., and was then charged with n-heptane (1042 L) while maintaining the temperature at 40° C. The solution was seeded with (2S,3S)-2-acetoxy-3-(3-acetoxy-2-methyl-benzoylamino)-4-phenyl-butyric acid (0.1 kg), and additional n-heptane (437 L) was then added slowly. The crystallizing mixture was maintained at 40° C. for 1 h. Additional n-heptane (175 L) was added while maintaining the temperature at 40° C. The crystalline suspension was cooled and held at 25° C. for 1 h, then at 0° C. for 2 h. The suspension was filtered, using n-heptane for rinsing. The wet cake was dried under vacuum at 55° C. to give 174 kg (74.5%) of (2S,3S)-2-acetoxy-3-(3-acetoxy-2-methyl-benzoylamino)-4-phenyl-butyric acid as a white solid: m.p.=152-154° C.; ¹H NMR (300 MHz, CDCl₃) 7.21-7.35 (m, 5H), 7.13 (app t, J=7.9 Hz, 1H), 7.01 (app d, J=8.1 Hz, 1H), 6.94 (app d, J=7.2 Hz, 1H), 5.99 (d, J=9.0 Hz, 1H), 5.33 (d, J=4.1 Hz, 1H), 4.96-5.07 (m, 1H), 3.07 (dd, J=5.5, 14.6 Hz, 1H), 2.90 (dd, J=10.0, 14.5 Hz, 1H), 2.30 (s, 3H), 2.18 (s, 3H), 1.96 (s, 3H); ¹³C NMR (125 MHz, CDCl₃) δ 170.4, 170.2, 169.6, 169.5, 149.5, 137.81, 136.5, 129.2, 128.6, 128.4, 127.0, 126.6, 124.5, 123.7, 73.1, 50.9, 35.9, 20.6, 20.5, 12.4; elemental analysis calcd for C₂₂H₂₃NO₇: C, 63.92; H, 5.61; N, 3.39; found: C, 64.22; H, 5.68; N, 3.33; MS (Cl) m/z 414.1572 (414.1553 calcd for C₂₂H₂₄NO₇, M+H⁺).

### Example 46

### Preparation of (4R)-3-[(2S,3S)-2-hydroxy-3-(3-hydroxy-2-methyl-benzoylamino)-4-phenyl-butyryl]-5,5-dimethyl-thiazolidine-4-carboxylic Acid Allylamide

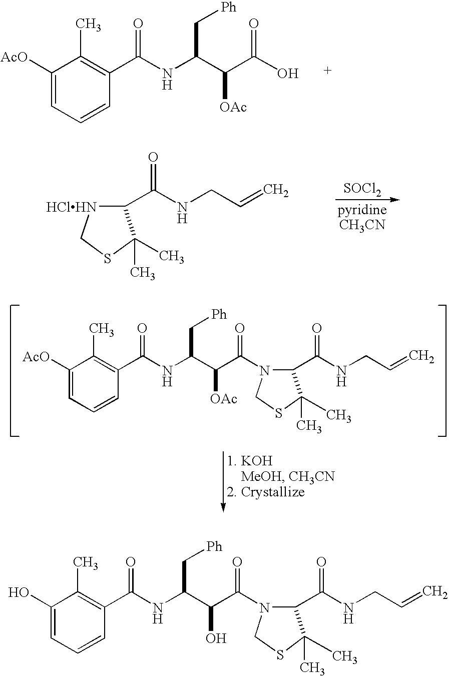

A solution of (2S,3S)-2-acetoxy-3-(3-acetoxy-2-methyl-benzoylamino)-4-phenyl-butyric acid (140 kg, 339 mol), CH₃CN (560 L), and pyridine (64.3 kg, 813 mol) was cooled to 15° C. SOCl₂ (44.3 kg, 373 mol) was charged while maintaining the temperature at 15° C. The mixture was held at 15° C. for 1 h. A separate reactor was charged with (R)-5,5-dimethyl-thiazolidine-4-carboxylic acid allylamide hydrochloride (96.6 kg, 408 mol), CH₃CN (254 L), and pyridine (29.5 kg, 373 mol), and was then cooled to 15° C. The (2S,3S)-2-acetoxy-3-(3-acetoxy-2-methyl-benzoylamino)-4-phenyl-butyric acid chloride solution was added to the (R)-5,5-dimethyl-thiazolidine-4-carboxylic acid allylamide solution, while maintaining the temperature at 15° C. The mixture was held at 15° C. for 6 h. A separate reactor was charged with KOH (167 kg, 2709 mol) and methanol (280 L) using a 0° C. cooling jacket. The resulting KOH/methanol solution was cooled to 5° C. The crude acetic acid 3-{(1S,2S)-2-acetoxy-3-[(R)-4-allylcarbamoyl-5,5-dimethyl-thiazolidin-3-yl]-1-benzyl-3-oxo-propylcarbamoyl}-2-methyl-phenyl ester mixture was added to the KOH/methanol solution while maintaining the temperature at 10° C. After addition was complete, the mixture was held at 25° C. for 3 h. The mixture was charged with H₂O (840 L) and ethyl acetate (840 L), and was then followed by acidification to pH 5-6.5 with concentrated HCl (85 kg) while maintaining the temperature at 20° C. The resulting layers were separated. The organic fraction was sequentially washed with 6.8 wt. % aqueous NaHCO₃ (770 L), an aqueous HCl/NaCl solution (H₂O: 875 L; conc. HCl: 207 kg; NaCl: 56 kg), 8.5 wt. % aqueous NaHCO₃ (322 L), and then with 3.8 wt. % aqueous NaCl (728 L). The resulting organic fraction was partially concentrated by distillation at one atmosphere. The solvent was exchanged with ethyl acetate by continuing distillation and maintaining the pot temperature at ≧70° C. Ethyl acetate was added such that the pot volume remained at approximately 840 L. The solution was then cooled to 20° C. and held at this temperature until crystallization was observed. n-Heptane (280 L) was added and the suspension was agitated at 15° C. for 4 h. The crystals were, using cold 2.4:1 (v/v) ethyl acetate/n-heptane for rinsing. The wet cake was dried under vacuum at 45° C. to provide crude (R)-3-[(2S,3S)-2-hydroxy-3-(3-hydroxy-2-methyl-benzoylamino)-4-phenyl-butyryl]-5,5-dimethyl-thiazolidine-4-carboxylic acid allylamide. Decolorization and recrystallization was conducted as follows: A mixture of crude (R)-3[(2S,3S)-2-hydroxy-3-(3-hydroxy-2-methyl-benzoylamino)-4-phenyl-butyryl]-5,5-dimethyl-thiazolidine-4-carboxylic acid allylamide, ADP carbon (21 kg), Supercel (3 kg), and ethyl acetate (780 L) was heated to 70° C. CH₃OH (40 L) was added to the mixture. The mixture was filtered, and the resulting clear filtrate was heated to reflux at one atmosphere to begin distillation. CH₃OH was displaced as follows: ethyl acetate (388 L) was charged while maintaining the pot volume at approximately 840 L and at 70° C. The solution was slowly charged with n-heptane (316 L), while maintaining a temperature of 70° C. The mixture was then cooled to 20° C. and was held at this temperature for 4 h. The crystals were filtered, using cold 2.1:1 (v/v) ethyl acetate/n-heptane for rinsing. The wet cake was dried under vacuum at 45° C. to give 103 kg (59.6%) of (4R)-3-[(2S,3S)-2-hydroxy-3-(3-hydroxy-2-methyl-benzoylamino)-4-phenyl-butyryl]-5,5-dimethyl-thiazolidine-4-carboxylic acid allylamide as a white crystalline solid: mp=173-175° C., ¹H NMR (300 MHz, DMSO-d₆) displayed a ˜10:1 mixture of rotamers, major rotamer resonances δ 9.35 (s, 1H), 8.04-8.15 (m, 2H), 7.13-7.38 (m, 5H), 6.96 (t, J=7.7 Hz, 1H), 6.79 (d, J=7.2 Hz, 1H), 6.55 (d, J=7.5 Hz, 1H), 5.71-5.87 (m, 1H), 5.45 (br d, J=6.2 Hz, 1H), 4.98-5.27 (m, 4H), 4.38-4.52 (m, 3H), 3.58-3.86 (m, 2H), 2.68-2.90 (m, 2H), 1.84 (s, 3H), 1.52 (s, 3H), 1.37 (s, 3H) [characteristic minor rotamer resonances δ 9.36 (s), 8.21 (d, J=10.5 Hz), 7.82 (5, J=5.8 Hz), 4.89 (s), 4.78 (AB q, J_{AB}=9.8 Hz, Δv=27.1 Hz), 4.17-4.24 (m), 2.93-3.01 (m), 1.87 (s), 1.41 (s)]; ¹³C NMR (75 MHz, DMSO-d₆) displayed a ˜10:1 mixture of rotamers, major rotamer resonances δ 170.4, 169.5, 168.2, 155.7, 139.6, 139.4, 135.5, 135.4, 129.9, 128.2, 126.2, 126.1, 121.9, 117.8, 115.6, 72.4, 72.1, 53.1, 51.4, 48.2, 41.3, 34.2, 30.5, 25.0, 12.6 [characteristic minor rotamer resonances δ 171.4, 169.7, 168.6, 139.0, 129.5, 128.4, 70.6, 54.2, 49.1, 41.5, 31.4, 24.8]; MS (Cl) m/z 512.2224 (512.2219 calcd for C₂₇H₃₄N₃O₅S, M+H+), elemental analysis calcd for C₂₇H₃₃N₃O₅S: C, 63.38; H, 6.50; N, 8.22; found: C, 63.19; H, 6.52; N, 8.10.

### Example 47

### Preparation of (2S,3S)-3-Amino-2-hydroxy-4-phenyl-butyric Acid; Hydrochloride

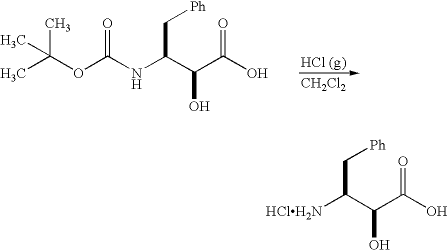

HCl gas (51 g, 1.4 mol) was bubbled into a suspension of (2S,3S)-3-tert-butoxycarbonylamino-2-hydroxy-4-phenyl-butyric acid (163 g, 551 mmol) and CH₂Cl₂ (2.0 L) at 0° C. The resulting off-white suspension was allowed to warm to ambient temperature and stir overnight. ¹H NMR analysis of a concentrated aliquot showed approximately 95% conversion to product. The suspension was cooled to 0° C., and additional HCl gas (46 g, 1.3 mol) was bubbled into the suspension. After warming to ambient temperature, the suspension was stirred overnight. The suspension was vacuum-filtered, the solid was rinsed with CH₂Cl₂ (200 mL), and the solid was then dried in a vacuum oven at 45° C. for 24 h to give 129 g (100%) of (2S,3S)-3-amino-2-hydroxy-4-phenyl-butyric acid; hydrochloride as a white solid: ¹H NMR (300 MHz, DMSO-d₆) δ 13.05 (br s, 1H), 8.25 (br s, 3H), 7.22-7.34 (m, 5H), 4.41 (d, J=2.6 Hz, 1H), 3.66 (br s, 1H), 2.84 (AB portion of ABX, J_{AX}=11.0 Hz, J_{BX}=2.8 Hz, Δv=19.6 Hz, 2H); ¹³C NMR (75 MHz, DMSO-d₆) d 172.4, 136.6, 129.8, 128.7, 127.1, 69.6, 55.0, 33.6; MS (Cl) m/z 196.0979 (196.0974 calcd for C₁₀H₁₄NO₃, M-Cl⁻).

### Example 48

### Preparation of (2S,3S)-3-(3-Acetoxy-2-methyl-benzoylamino)-2-hydroxy-4-phenyl-butyric Acid

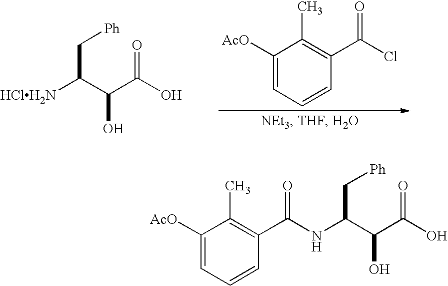

NEt₃ (186 mL, 1.34 mol) was added to a suspension of (2S,3S)-3-amino-2-hydroxy-4-phenyl-butyric acid; hydrochloride (100 g, 432 mmol), H₂O (320 mL), and tetrahydrofuran (320 mL). The suspension was cooled to 4° C. and a solution of acetic acid 3-chlorocarbonyl-2-methyl-phenyl ester (93.6 g, 440 mmol) and THF (160 mL) was added dropwise. The resulting solution was warmed to ambient temperature and stir for 1 h. The solution was cooled to 10° C. and the pH was adjusted to 2.0 using 6 N HCl (87 mL). NaCl (25 g) and tetrahydrofuran (200 mL) were added, and the mixture was warmed to ambient temperature. The phases were separated and the tetrahydrofuran fraction was dried over MgSO₄ and filtered. The filtrate was concentrated to a volume of 330 mL using a rotary evaporator, and was then diluted with tetrahydrofuran (230 mL). n-Heptane (1.2 L) was added slowly and the resulting white suspension of solid was stirred at ambient temperature overnight. The suspension was vacuum-filtered, the solid was rinsed with n-heptane (2×500 mL), and the solid was dried in a vacuum oven at 45° C. for 24 h to give 150 g (93.6%) of (2S,3S)-3-(3-acetoxy-2-methyl-benzoylamino)-2-hydroxy-4-phenyl-butyric acid as a white solid that was contaminated with ˜7.7 mol % Et₃NeHCl: mp=189-191° C., ¹H NMR (300 MHz, DMSO-d₆) δ 12.65 (br s, 1H), 3.80 (d, J=9.7 Hz, 1H), 7.16-7.30 (m, 6H), 7.07 (dd, J=1.1, 8.0 Hz, 1H), 7.00 (dd, J=1.1, 7.5 Hz), 4.40-4.52 (m, 1H), 4.09 (d, J=6.0 Hz, 1H), 2.92 (app dd, J=2.9, 13.9 Hz, 1H), 2.76 (app dd, J=11.4, 13.9 Hz, 1H), 2.29 (s, 3H), 1.80 (s, 3H); ¹³C NMR (75 MHz, DMSO-d₆) δ 174.4, 169.3, 168.1, 149.5, 139.7, 139.4, 129.5, 128.3, 127.9, 126.5, 126.3, 124.8, 123.3, 73.2, 53.5, 35.4, 20.8, 12.6; MS (Cl) m/z 372.1464 (372.1447 calcd for C₂₀H₂₂NO₆, M+H⁺).

1. Amorphous (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof.

2. A pharmaceutical composition comprising amorphous (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof.

3. A pharmaceutical composition according to claim 2, wherein at least about 5 wt % of the total amount of (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide present is in an amorphous form.

4. A pharmaceutical composition according to claim 2, further comprising a matrix.

5. A pharmaceutical composition according to claim 4, wherein said matrix is selected from at least one of an ionizable cellulosic polymer, a nonionizable cellulosic polymer, and a noncellulosic polymer.

6. A pharmaceutical composition according to claim 5, wherein said matrix is selected from at least one ionizable cellulosic polymer.

7. A pharmaceutical composition according to claim 6, wherein said at least one ionizable cellulosic polymer is selected from hydroxypropyl methyl cellulose acetate succinate, carboxymethyl ethyl cellulose, cellulose acetate phthalate, hydroxypropyl methyl cellulose phthalate, methyl cellulose acetate phthalate, cellulose acetate trimellitate, hydroxypropyl cellulose acetate phthalate, hydroxypropyl methyl cellulose acetate phthalate, cellulose acetate terephthalate and cellulose acetate isophthalate, and mixtures thereof.

8. A pharmaceutical composition according to claim 5, wherein said matrix is selected from at least one nonionizable cellulosic polymer.

9. A pharmaceutical composition according to claim 8, wherein said at least one nonionizable cellulosic polymer is selected from hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose, and mixtures thereof.

10. A pharmaceutical composition according to claim 5, wherein said matrix is selected from at least one noncellulosic polymer.

11. A pharmaceutical composition according to claim 10, wherein said at least one noncellulosic polymer is selected from carboxylic acid functionalized polymethacrylates, carboxylic acid functionalized polyacrylates, amine-functionalized polyacrylates, amine-functionalized polymethacrylates, proteins, carboxylic acid functionalized starches, vinyl polymers and copolymers having at least one substituent selected from the group consisting of hydroxyl, alkylacyloxy, and cyclicamido, vinyl copolymers of at least one hydrophilic, hydroxyl-containing repeat unit and at least one hydrophobic, alkyl- or aryl-containing repeat unit, polyvinyl alcohols that have at least a portion of their repeat units in the unhydrolyzed form, polyvinyl alcohol polyvinyl acetate copolymers, polyethylene glycol polypropylene glycol copolymers, polyvinyl pyrrolidone, polyethylene polyvinyl alcohol copolymers, polyoxyethylene-polyoxypropylene block copolymers and mixtures thereof.

12. A pharmaceutical composition according to claim 4, wherein said composition is in the form of a solid amorphous dispersion.

13. A pharmaceutical composition according to claim 12, wherein said solid amorphous dispersion is substantially homogeneous.

14. A pharmaceutical composition according to claim 12, wherein said solid amorphous dispersion comprises at least about 40 wt % of (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof.

15. A pharmaceutical composition according to claim 14, wherein said solid amorphous dispersion comprises at least 70 wt % of (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof.

16. A pharmaceutical composition according to claim 15, wherein said solid amorphous dispersion comprises at least 90 wt % of (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof.

17. A pharmaceutical composition, comprising (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof, that when administered to an aqueous use environment, provides at least one of:
(a) a maximum dissolved concentration of (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in said use environment that is at least 1.25-fold that provided by a control composition; and
(b) a concentration of said (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in said use environment versus time area under the curve (AUC) for any period of at least 90 minutes between the time of introduction into said use environment and about 270 minutes following introduction to said use environment that is at least 1.25-fold that of said control composition;
wherein said control composition consists essentially of an equivalent quantity of said (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide in crystalline Form I alone.

18. A pharmaceutical composition according to claim 17, wherein said aqueous use environment consists essentially of:
(a) 20 mM Na₂HPO₄;
(b) 47 mM KH₂PO₄;
(c) 87 mM NaCl;
(d) 0.2 mM KCl;
(e) at pH 6.5;
(f) 290 mOsm/kg; and
(e) at a temperature of 37° C.;
wherein the total amount of said use environment is about 1.8 mL and the amount of (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide used is such that the total concentration of (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide would have been 3000 μg/mL if it had all dissolved.

19. A pharmaceutical composition according to claim 17, wherein at least about 5 wt % of the total amount of (4R)—N-allyl-3-{(2S,3S)-2-hydroxy-3-[(3-hydroxy-2-methylbenzoyl)amino]4-phenylbutanoyl}-5,5-dimethyl-1,3-thiazolidine-4-carboxamide present is in an amorphous form.

20. A method of treating an HIV-infected mammal, comprising administering to said mammal a pharmaceutical composition comprising an HIV replication-inhibiting amount of amorphous (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof.

21. A method according to claim 20, wherein said pharmaceutical composition further comprises a matrix.

22. A method according to claim 21, wherein said composition is in the form of a solid amorphous dispersion.

23. A method according to claim 21, wherein said matrix is selected from at least one of an ionizable cellulosic polymer, a nonionizable cellulosic polymer, and a noncellulosic polymer.

24. A method according to claim 23, wherein said matrix is selected from at least one ionizable cellulosic polymer.

25. A method according to claim 24, wherein said at least one ionizable cellulosic polymer is selected from hydroxypropyl methyl cellulose acetate succinate, carboxymethyl ethyl cellulose, cellulose acetate phthalate, hydroxypropyl methyl cellulose phthalate, methyl cellulose acetate phthalate, cellulose acetate trimellitate, hydroxypropyl cellulose acetate phthalate, hydroxypropyl methyl cellulose acetate phthalate, cellulose acetate terephthalate and cellulose acetate isophthalate, and mixtures thereof.

26. A method according to claim 23, wherein said matrix is selected from at least one nonionizable cellulosic polymer.

27. A method according to claim 26, wherein said at least one nonionizable cellulosic polymer is selected from hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose, and mixtures thereof.

28. A method according to claim 23, wherein said matrix is selected from at least one noncellulosic polymer.

29. A method according to claim 28, wherein said at least one noncellulosic polymer is selected from carboxylic acid functionalized polymethacrylates, carboxylic acid functionalized polyacrylates, amine-functionalized polyacrylates, amine-fuctionalized polymethacrylates, proteins, carboxylic acid functionalized starches, vinyl polymers and copolymers having at least one substituent selected from the group consisting of hydroxyl, alkylacyloxy, and cyclicamido, vinyl copolymers of at least one hydrophilic, hydroxyl-containing repeat unit and at least one hydrophobic, alkyl- or aryl-containing repeat unit, polyvinyl alcohols that have at least a portion of their repeat units in the unhydrolyzed form, polyvinyl alcohol polyvinyl acetate copolymers, polyethylene glycol polypropylene glycol copolymers, polyvinyl pyrrolidone, polyethylene polyvinyl alcohol copolymers, polyoxyethylene-polyoxypropylene block copolymers and mixtures thereof.

30. A process for preparing a pharmaceutical composition comprising:
(a) dissolving a compound in a spray solution comprising a solvent; and
(b) rapidly evaporating said solvent from said spray solution to afford an amorphous form of said compound;
wherein said compound is (4R)—N-allyl-3-{(2S,3S)-2-hydroxy-3-[(3-hydroxy-2-methylbenzoyl)amino]-4-phenylbutanoyl}-5,5-dimethyl-1,3-thiazolidine-4-carboxamide, or a pharmaceutically acceptable salt or solvate thereof.

31. The process of claim 30, wherein said spray solution further comprises a matrix.

32. The process of claim 31, wherein said matrix comprises at least one polymer selected from an ionizable cellulosic polymer, a nonionizable cellulosic polymer, and a noncellulosic polymer.

33. The process of claim 32, wherein said matrix is selected from the group consisting of hydroxypropyl methyl cellulose acetate, hydroxypropyl methyl cellulose, hydroxypropyl cellulose, methyl cellulose, hydroxyethyl methyl cellulose, hydroxyethyl cellulose acetate, hydroxyethyl ethyl cellulose, hydroxypropyl methyl cellulose acetate succinate, carboxymethyl ethyl cellulose, cellulose acetate phthalate, hydroxypropyl methyl cellulose phthalate, hydroxypropyl methyl cellulose acetate phthalate, methyl cellulose acetate phthalate, cellulose acetate trimellitate, hydroxypropyl cellulose acetate phthalate, cellulose acetate terephthalate and cellulose acetate isophthalate.

34. The process of claim 30, wherein said solvent is selected from the group consisting of methanol and mixtures of methanol and water.

35. The product of the process according to claim 30.
